(19) United States
(12) Patent Application Publication
(10) Pub. No.: US 2025/0294957 A1
(43) Pub. Date: Sep. 18, 2025

(54) EMITTING MATERIALS FOR ELECTROLUMINESCENT DEVICES AND OTHER APPLICATIONS

(71) Applicant: Kebotix, Inc., Cambridge, MA (US)

(72) Inventors: Semion Saykin, Arlington, MA (US); Victoria Campbell, Somerville, MA (US); Selma Duhovic, Denver, CO (US); David Lee Wheeler, Brighton, MA (US); Kevin Ryan, Watertown, MA (US); Sreeletha Joby Eldo, Andover, MA (US); Nathan Darrell Peterson Ricke, Medford, MA (US); Zhongwei Hu, Newton Upper Falls, MA (US); Dennis Sheberla, Bedford, MA (US); Jhaylen McDavid, Cambridge, MA (US)

(73) Assignee: Kebotix, Inc., Cambridge, MA (US)

(21) Appl. No.: 18/722,064

(22) PCT Filed: Dec. 20, 2022
(86) PCT No.: PCT/US2022/053522

§ 371 Date: Jun. 20, 2024

Related U.S. Application Data
(60) Provisional application No. 63/292,276, filed on Dec. 21, 2021.

(51) Int. Cl. H10K 50/12; C09K 11/06; H10K 85/60

(52) U.S. Cl. CPC H10K 50/12; C09K 11/06; H10K 85/615; H10K 85/656; H10K 85/6574; C09K 2211/1033; C09K 2211/1037

(57) ABSTRACT

The present disclosure generally relates to emitting organic molecules, including but not limited to, thiazoles such as 1,3-thiazoles, 4-methyl-1,3-thiazoles, etc. In some cases, the molecules are able to emit in blue or green wavelengths. In some aspects, such molecules may be used in organic light emitting diodes. In certain embodiments, the molecules may comprise one or more bridge moieties and one or more chromophores in certain cases. In some embodiments, these molecules can be used as electroluminescent media in devices requiring light emission as a function of applied voltage. Other embodiments of the disclosure are generally directed to systems and devices using such molecules, methods of using such molecules, e.g., to control the emission of light, kits involving such molecules, or the like.

### RELATED APPLICATIONS

This application claims the benefit of U.S. Provisional Patent Application Ser. No. 63/292,276, filed Dec. 21, 2021, entitled “Emitting Materials for Electroluminescent Devices and Other Applications,” incorporated herein by reference in its entirety.

### GOVERNMENT FUNDING

This invention was made with government support under Grant. No. DE-SC0020762 awarded by the Department of Energy. The government has certain rights in the invention.

### FIELD

The present disclosure generally relates to emitting organic molecules, including but not limited to, thiazoles such as 1,3-thiazoles, 4-methyl-1,3-thiazoles, etc. In some cases, the molecules are able to be excited by electric potential and may emit light in the visible spectrum.

### BACKGROUND

Organic light-emitting diode (OLED) are light-emitting diodes (LED) in which an organic compound is placed between two conductors and shows electroluminescence in response to an excitation such as an electric current. For example, when a voltage is applied, an OLED material may emit light of a given wavelength. Such materials may be used in a variety of applications such as displays for televisions, computer screens, and smart phones; in solid-state lighting such as outdoor or indoor luminaires, or automotive luminaires, as well as in smart labels and wearable devices. However, as many OLED materials exhibit limited spectral ranges and stability, there remains a need for new types of OLED materials for various applications. This point is particularly true for blue electroluminescent compounds due to their intrinsically low electrochemical stability.

OLED materials typically work through the recombination of electrons and holes in a host transport material that cause radiative decay of the molecular excited levels. This excitation results in interactions between electrons and holes that splits the energy levels into dark triplets and bright singlets.

Conventional OLEDs have a maximum theoretical efficiency of 25% due to the recombination of electrons and holes that affords a statistical mixture of four spin states (one singlet and three triplet sublevels). OLED material design has primarily focused on finding ways to harvest the energy of the dark triplet states into usable emissive states. However, colors such as blue, violet, and cyan require higher energy excited states that facilitated the degradation process of the OLED. Accordingly, improvements are needed.

### SUMMARY

The present disclosure generally relates to emitting organic molecules, including but not limited to, thiazoles such as 1,3-thiazoles, 4-methyl-1,3-thiazoles, etc. In some cases, the molecules are able to be excited by electric potential and may emit light in the visible spectrum. The subject matter of the present disclosure involves, in some cases, interrelated products, alternative solutions to a particular problem, and/or a plurality of different uses of one or more systems and/or articles.

Certain embodiments are generally directed to certain classes of emitting organic molecules that can be used in OLEDs (organic light-emitting diodes) or other applications. These include certain classes of thiazole-bridged molecules. For example, some embodiments provide systems and methods for preparing blue and/or green OLEDs.

In some embodiments, the organic molecules comprise two or more chromophores bridged by a thiazole moiety at two or three of the carbon positions. The “bridge” generally refers to a molecular fragment that may be pi (π)-conjugated, and can be covalently linked between two or more chromophore moieties. The chromophore moieties may include a “donor” fragment that is likely to donate electron from its highest occupied molecular orbital (HOMO), and an “acceptor” fragment that is likely accept electron in its lowest unoccupied molecular orbital (LUMO). Without wishing to be bound by any theory, it is believed that the bridge moiety can enhance fluorescence from the molecule, may impart steric rigidity to the molecule, and/or may restrict the chromophores moieties (e.g., the donor and acceptor moieties) into specific configurations. This restriction, in turn, may lead to delocalization of electronic states in the linked chromophores, and/or may prevent the overlap between the conjugated pi (π) system of the chromophore moieties. Examples of suitable bridge molecules include, but are not limited to, phenyl, biphenyl, 2,7-fluorenyl-, 3,6-(9(H)-carbazoyl), 1,3,5-triazinyl-, furyl, thienyl, pyridyl and others.

In addition, in some embodiments, the organic molecules may be electroluminescent. In some cases, the organic molecules can be compatible with vapor-deposition and/or solution-processed OLED technologies.

One aspect is generally drawn to an electroluminescent device. According to one set of embodiments, the electroluminescent device comprises an electroluminescent region comprising a 1,3-thiazole, and a voltage source able to apply voltage to the electroluminescent region.

In another set of embodiments, the electroluminescent device comprises a cathode, an anode, a voltage source electrically connecting the anode and the cathode, and an electroluminescent region comprising a 1,3-thiazole, positioned between cathode and anode.

Another aspect is generally drawn to a method. In accordance with one set of embodiments, the method comprises applying voltage to an electroluminescent material comprising a 1,3-thiazole to cause the electroluminescent material to exhibit light emission. In another set of embodiments, the method comprises applying voltage to an electroluminescent material comprising a molecule selected from FIGS. 10-23 to cause the electroluminescent material to emit light.

In yet another set of embodiments, the method comprises applying voltage to an electroluminescent material comprising a molecule to cause the electroluminescent material to emit light, where the molecule is selected from the group consisting of the following molecules, each identified by a first hash block of an InChIKey representation of the molecule: AAIFIJHQPULVMI, ZEXBHUPLNIGHEZ, QOLRRWCRWYQMLR, GXVWHVFJXJITDO, XBWCSDLHQPFVRP, FTOBIPSLLFQWGQ, XUDAQKXVVYVOMW, SWXMNBXTODAPJU, IMUZIXSBGCDBDO, LFUMMFVQFZGISH, LFUMMFVQFZGISH, OWGMSZXTMKECCI, FVKPJEPVXUOALV, DQIFIRZAHLHCSV, UYUUIVYOLSYCML, XGQROYXDUMHMKQ, GDGVBBPMHUAWER, SWUVVXLGXKQWOK, DKGOKAARXPCDJB, AQSPXOQHZFBFDI, BXZOOIPJUSPRDW, FSDQOJYTVJRTHL, YUEYOBZWLODUGC, GBQCXTMGESMUEL, OGKZAJYSJBFSOG, QTGAIVYNKJBHEJ, GAOSHNRBJWVZDA, HNWAWCPRDIBDDE, WOFQROVFBXYBOZ, HHVBGAXPRFMPGS, CTBHRVFXOWLCLT, SNPNGHYHZNWCPM, IFCNOEBWGRXPBH, CZFNZPVICSEKKV, BQGVNJWCBFSZQK, ZRNUAICMPZORBH, APFNLFNUHQLJDG, JHTGJOZYPSMACV, LNACLYIJOUNWRW, LNPNEMBJXUSTRM, UNHOUQALCJXAAY, QDOVKCKZAFNXEC, GXVIHPNZPLMOFI, LTMQBXYXRIZMLG, CGRRZMMRRFNWPW, PTEIMXYLBHMYET, OXGRFYSZCWOZFS, DSWWUPRZCBKXLE, OYAZGLJGAVEGBZ, IMTDFCZBMXEELU, WOTFSSPRRQWLBU, VWEXHTKBKCUGLB, HKALLZZMGBMRGJ, DEVSOZBTEMFORY, DHMAFZYIVUIGJM, IBFRLZWMQUPRPR, CTCKQLXMUVOIOU, RFFDKFSLLAFKMM, FEKZKTRIFUBKER, DVQPXNLTBBDOIZ, JUOGGWLHDVNOBX, IBYTUCBHKIXOIW, ZWDQNGQLKSREND, VISGKGWXPGXKBL, UARGLLSVBPBWMS, QCBIHGXCUPKGRY, RPOBMJZIHYTRAU, HFYABEGBJVTUML, FFALRSFIGCORHO, ULRNJTPMFKQCMO, HZYZTHMJYSLSMJ, JUBQNCLMFCHMQN, DJRPCUJSQLWPAZ, SRJYATBZLBEBJY, XPGJQECYTKNGRN, NJWNZBKTAPHFTH, BEMNWKLFFWUKDU, SILYRWRXVMHRTM, YDYHMDFVQIVLML, ZOMAJIOFMSVVNT, ISYKKWICBXSHKY, LOPXOGLVYSLSGC, SPZYRPPRLBUQAH, VYJVFVAIWLSCED, ZDCQXVUZVLQKLY, JHQCZXRSVLVYOW, KMGAKZISGVDXGW, BGYXIGIIRVQASS, YNAZTCWGRIOKCB, FFLDFNQQENULNK, PBIPJNJUZRAYHE, KIPVEMHJQKNZFL, LDYKAHFEBNQUGJ, VJTJFGNPPUSEQE, IELYXTSOHBZECP, ISCMOHOLBLISMQ, RRYUMHOBKQOGAD, PFQCMMXHKKNLOT, VIMFKWUTJUEQJK, POBQTDRNVWERPF, CFSSKPPFJCBYMY, FWVIVAJNBPEESS, JROPTVRWUXYQMR, FCLLWHJYNSGBMQ, PHXINTIMPBVAIX, JBJUKSOOTJPZHH, ICYVZGRFKOFTAD, PWMMHOOXMFCACY, UGVPJXSWPBOMJP, QLXCDVXTOMYAMU, QIBGZDFTXWBTMI, RLDSMSDTKCVPRO, KMHRNTKTYZADTN, BMSBKLICNZLUBE, HXTPVMBISBGNAC, JHLRLFFIUWGHDV, VLBHFTWJPSDFIE, NCXAIOZZQAVZMI, SVKUUBRDEQXQLR, BJUWJXPRDCKMNI, IZRFGTAUUUKGTM, DAJDHRGFWQVASM, BHXHUNWNDBGQMO, XKCKUCWGKCCQGV, KWHAUGPQNZAJDS, NEZXCGPTAYIZCU, GICLYTUDYTXFRS, PRHUAJBGRRUGHJ, PASWKTPBVDOBBE, GIXSXCOUMMISFJ, YUCWUNZRDPXDNR, HBZINHRGTVCNDG, NBTNJXPNOBISRV, VPRSWFQXIXZSBI, ICYBHLYOCZWBFD, ABPFDSNHUAFYPV, RALCBWCBMXROBW, VGFMNTFZYOFFLG, YWQMKNAAENETQK, HYQHOTXHFKNJHU, ZXVYYXLJDXCZCI, MXWPXHCLXWQNNW, SHJHMYRRZLUUKL, JYSSHXMOBVYXSS, RAQWJQONIVFTFW, GSXZETKDCIEKIE, PASWKTPBVDOBBE, GIXSXCOUMMISFJ, YUCWUNZRDPXDNR, HBZINHRGTVCNDG, NBTNJXPNOBISRV, VPRSWFQXIXZSBI, ICYBHLYOCZWBFD, ABPFDSNHUAFYPV, RALCBWCBMXROBW, VGFMNTFZYOFFLG, YWQMKNAAENETQK, HYQHOTXHFKNJHU, ZXVYYXLJDXCZCI, MXWPXHCLXWQNNW, SHJHMYRRZLUUKL, JYSSHXMOBVYXSS, RAQWJQONIVFTFW, GSXZETKDCIEKIE, MHLSVUZSMRYLIZ, ZQBXLMSVNMMTCS, KEEVEQZIGAQZKE, WOMDNAPZTGCNJL, ADNZGPRKIJKESU, QXEMFQGQZKRSTJ, SOAPJSITMYDWDL, GKCJZXSGGINQDD, NIDRNHQAGJOWFJ, VJMBREBKNLAOIZ, TVUGXXXCGDLQBU, AECIIKUVLSLILY, TZIAQFUWVSWNHI, and CUDMJKXXXZXZGS.

Yet another aspect is generally drawn to certain molecules. In one set of embodiments, the molecules may be selected from the group consisting of the following molecules, each identified by a first hash block of an InChIKey representation of the molecule: QOLRRWCRWYQMLR, GXVWHVFJXJITDO, XBWCSDLHQPFVRP, FTOBIPSLLFQWGQ, XUDAQKXVVYVOMW, SWXMNBXTODAPJU, IMUZIXSBGCDBDO, LFUMMFVQFZGISH, LFUMMFVQFZGISH, OWGMSZXTMKECCI, FVKPJEPVXUOALV, DQIFIRZAHLHCSV, UYUUIVYOLSYCML, XGQROYXDUMHMKQ, GAOSHNRBJWVZDA, VISGKGWXPGXKBL, SPZYRPPRLBUQAH, KIPVEMHJQKNZFL, LDYKAHFEBNQUGJ, VJTJFGNPPUSEQE, IELYXTSOHBZECP, ISCMOHOLBLISMQ, ABPFDSNHUAFYPV, JYSSHXMOBVYXSS, ICYBHLYOCZWBFD, GSXZETKDCIEKIE, GKCJZXSGGINQDD, CUDMJKXXXZXZGS, AAIFIJHQPULVMI, and ZEXBHUPLNIGHEZ.

In another aspect, the present disclosure encompasses methods of making one or more of the embodiments described herein, such as 1,3-thiazoles or 4-methyl-1,3-thiazoles. In still another aspect, the present disclosure encompasses methods of using one or more of the embodiments described herein, such as 1,3-thiazoles or 4-methyl-1,3-thiazoles.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates an electroluminescent device in accordance with one embodiment of the disclosure;

FIG. 2 illustrates a structure in accordance with one embodiment;

FIGS. 3A-3E illustrate various properties of the compound shown in FIG. 2, in yet another embodiment;

FIG. 4 illustrates an electroluminescent device using the compound shown in FIG. 2, in accordance with another embodiment;

FIGS. 5A-5C illustrate certain molecular structures, in yet another embodiment;

FIG. 6 illustrates another structure, in accordance with another embodiment;

FIGS. 7A-7E illustrate electroluminescence spectrum and JVL curves for the compound shown in FIG. 6, in yet another embodiment;

FIG. 8 illustrates an electroluminescent device using the compound shown in FIG. 6, in accordance with another embodiment;

FIG. 9 illustrate certain molecular structures, in yet another embodiment; and

FIGS. 10-23 illustrate certain additional molecules, in accordance with other embodiments.

### DETAILED DESCRIPTION

The present disclosure generally relates to emitting organic molecules, including but not limited to, thiazoles such as 1,3-thiazoles, 4-methyl-1,3-thiazoles, etc. In some cases, the molecules are able to emit in blue or green wavelengths. In some aspects, such molecules may be used in organic light emitting diodes. In certain embodiments, the molecules may comprise one or more bridge moieties and one or more chromophores in certain cases. In some embodiments, these molecules can be used as electroluminescent media in devices requiring light emission as a function of applied voltage. Other embodiments of the disclosure are generally directed to systems and devices using such molecules, methods of using such molecules, e.g., to control the emission of light, kits involving such molecules, or the like.

In addition, in certain embodiments, molecules such as those described herein may be electroluminescent, and/or may be useful in electroluminescent media. For instance, the molecules may be compatible with vapor-deposition and/or solution-processed OLED technologies. In one set of embodiments, for example, such molecules may be used in devices which can exhibit a change in light emission due to change in applied voltage. Examples of such devices include indoor or outdoor luminaires, automotive luminaires, displays, or the like.

OLEDs are light-emitting diodes in which the electroluminescent layer is a thin film comprising organic molecules that emit light in response to an electric current. In one embodiment, an OLED may be composed of an organic light-emitting molecule located between two electrodes, an anode and a cathode. The organic molecules may be selected to be conductive due to the delocalization of pi (π) electrons over part or all of the molecule, e.g., such that at least a portion of the molecule exhibits a conjugated system of p orbitals due to the pi electrons. When voltage is applied, positive charges may be introduced via the anode and/or negative charges may be introduced via the cathode. Electrostatic forces may bring the electrons and holes in proximity until they recombine and form an exciton. Radiation can be emitted at a frequency the visible spectrum due to the excited state decay and the subsequent relaxation of the energy levels of the electron. The frequency of this radiation may depend on the mechanism of radiation (e.g., fluorescence, S₁->S₀ or phosphorescence, T₁->S₀). However, thiazoles such as 1,3-thiazoles or 4-methyl-1,3-thiazoles have not been previously been identified as being suitable for OLEDs or similar applications.

Thus, one aspect as discussed herein is generally directed to systems and methods of electrically controlling the emission of light. In some embodiments, these may include molecules that can produce light in the blue or green region of the visible spectrum when they are excited via electric current. For example, in some cases, the molecules may comprise one or more conjugated groups and a thiazole (or similar) bridge moiety. Such optoelectronic molecules can be used in electroluminescent media, where the amount of light emission is controllable by applying voltages. For example, the optoelectronic molecule may exhibit light emission at varying biases. In some cases, a variety of different voltages can be applied to control the light emission of the electroluminescent media.

For instance, certain embodiments are generally directed to emitting organic molecules. In some embodiments, the molecules may comprise one or more bridge moieties and one or more chromophores in certain cases.

The molecules may comprise two or more chromophores bridged by a thiazole (or similar) bridge moiety. The bridge may be a pi (π)-conjugated moiety that can be covalently linked between two or more chromophore moieties. The donor may be a moiety that is able to donate an electron from its highest occupied molecular orbital (HOMO). The acceptor may be a moiety that is able to accept an electron in its lowest unoccupied molecular orbital (LUMO). Without wishing to be bound by any theory, it is believed that the bridge moiety can, in some embodiments, enhance fluorescence from the molecule, impart steric rigidity to the molecule, and/or restrict the donor and acceptor moieties into specific configurations. This may lead to delocalization of electronic states in the linked donor and acceptor and/or may prevent overlap between the conjugated pi systems of the donor and acceptor moieties.

Accordingly, some aspects of the present disclosure are generally directed to thiazoles such as 1,3-thiazoles, 4-methyl-1,3-thiazoles, etc. 1,3-thiazoles have not generally been identified as being suitable optoelectronic molecules, e.g., for use within electroluminescent media. However, it has been found that certain types of 1,3-thiazoles are able to absorb visible (400-700 nm) light and/or near-infrared light (e.g., wavelengths of 700-2500 nm) in response to applied voltages, and/or emit in blue or green wavelengths (e.g., wavelengths of 450-600 nm).

In addition, in some embodiments, certain types of 1,3-thiazoles are able to emit light at wavelengths of between 350 nm and 900 nm, or in the near-UV range (e.g., between 300 nm and 400 nm). In some cases, the 1,3-thiazoles are able to emit light at wavelengths of at least 300 nm, at least 350 nm, at least 400 nm, at least 450 nm, at least 500 nm, at least 550 nm, at least 600 nm, at least 650 nm, at least 700 nm, at least 750 nm, at least 800 nm, and/or no more than 900 nm, no more than 850 nm, no more than 800 nm, no more than 750 nm, no more than 700 nm, no more than 650 nm, no more than 600 nm, no more than 550 nm, no more than 500 nm, no more than 450 nm, no more than 400 nm, etc. Combinations of any of these ranges are also possible.

In one set of embodiments, for instance, a 1,3-thiazole may have a structure:

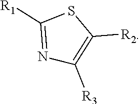

In some cases, the 1,3-thiazole may be a 4-methyl-1,3-thiazole, i.e., where R³ is a methyl.

In addition, in some embodiments, R₁, R₂ and R₃ may independently be —H or selected from an alkyl (such as a methyl group, an ethyl group, a propyl group, a butyl group or the like), a cycloalkyl (such as a cyclopropyl group, a cyclobutyl group or a cyclopentyl group, a cyclohexyl group, etc. or the like), an aromatic (such as phenyl group, a naphthyl group, an anthracene group, a fluorenyl group, or the like), a heterocyclic (such as an epoxide group, a furyl group, a pyrrolyl group, a thiazolyl group, an oxazolyl group, an imidazolyl group, a pyridyl group, a pyrazinyl group, a pyrimidinyl group, a piperidinyl group, a pyrazolyl group, etc.), a hydroxyl, an alkoxy (such as a methoxy, an ethoxy or similar), an amine such as —NH₂, an alkylamine (such as methyl amine, ethyl amine propyl amine or the like), a dialkylamine (such as dimethyl amine, diisopropyl amine, etc.) an arylamine (such as phenyl amine, naphthyl amine etc.), a diarylamine (such as diphenyl amine or the like), an amide such as —CONH₂, an N-substituted amide (where substituent is an alkyl or an aryl group or carboxylic acid or an alkoxy carbonyl group, such as a methoxy carbonyl group, an ethoxy carbonyl group, a phenoxy carbonyl etc.), an alkyl sulfonyl (such as a methyl sulfonyl group, an ethyl sulfonyl group, a p-tolyl sulfonyl group, a cyclohexyl sulfonyl group, etc.), a ketone (such as methyl ketone, an ethyl ketone or a phenyl ketone, etc.), a sulfonamide, a nitrile, a nitro, a halogen (for example, F, Cl, Br or I), a trifluoromethyl, a trichloromethyl, a tribromomethyl, an acyl, or the like.

In some embodiments, each of R₁, R₂, and R₃ may independently be an aromatic group such as a furan, an oxadiazole, a coumarin, a fluorene, or the like. In some cases, R₂, or R₃ may be —H, or may be selected from groups such as an alkyl group (methyl, ethyl, propyl, isopropyl, n-butyl, t-butyl, pentyl group, hexyl, octyl, dodecyl, etc.), a cycloalkyl group (such as a cyclopropyl, cyclopentyl, cyclohexyl, cyclooctyl, etc.), or the like. In some embodiments, one of R₂ or R₃ is either —H or a methyl.

For example, in one embodiment, the 1,3-thiazole may have a structure:

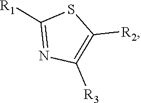

where R₁ may comprise a coumarin, and/or R₃ may comprise a fluorene. R₂ may be —H, or may be selected from groups such as an alkyl group (methyl, ethyl, propyl, isopropyl, n-butyl, t-butyl, pentyl group, hexyl, octyl, dodecyl, etc.), a cycloalkyl group (such as a cyclopropyl, cyclopentyl, cyclohexyl, cyclooctyl, etc.), or the like.

In some embodiments, R₁ may have a structure such as:

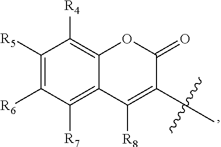

where R₄, R₅, R₆, R₇, and R₈ may each independently be —H or selected from an alkyl (such as a methyl group, an ethyl group, a propyl group, a butyl group or the like), a cycloalkyl (such as a cyclopropyl group, a cyclobutyl group or a cyclopentyl group, a cyclohexyl group, etc. or the like), an aromatic (such as phenyl group, a naphthyl group, an anthracene group, a fluorenyl group, or the like), a heterocyclic (such as an epoxide group, a furyl group, a pyrrolyl group, a thiazolyl group, an oxazolyl group, an imidazolyl group, a pyridyl group, a pyrazinyl group, a pyrimidinyl group, a piperidinyl group, a pyrazolyl group, etc.), a hydroxyl, an alkoxy (such as a methoxy, an ethoxy or similar), an amine such as —NH₂, an alkylamine (such as methyl amine, ethyl amine propyl amine or the like), a dialkylamine (such as dimethyl amine, diisopropyl amine, etc.) an arylamine (such as phenyl amine, naphthyl amine etc.), a diarylamine (such as diphenyl amine or the like), an amide such as —CONH₂, an N-substituted amide (where substituent is an alkyl or an aryl group or carboxylic acid or an alkoxy carbonyl group, such as a methoxy carbonyl group, an ethoxy carbonyl group, a phenoxy carbonyl etc.), an alkyl sulfonyl (such as a methyl sulfonyl group, an ethyl sulfonyl group, a p-tolyl sulfonyl group, a cyclohexyl sulfonyl group, etc.), a ketone (such as methyl ketone, an ethyl ketone or a phenyl ketone, etc.), a sulfonamide, a nitrile, a nitro, a halogen (for example, F, Cl, Br or I), a trifluoromethyl, a trichloromethyl, a tribromomethyl, an acyl, or the like.

In certain embodiments, R₃ may have a structure such as:

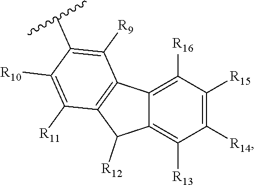

where R₉, R₁₀, R₁₁, R₁₂, R₁₃, R₁₄, R₁₅, and R₁₆ may each independently be —H or selected from an alkyl (such as a methyl group, an ethyl group, a propyl group, a butyl group or the like), a cycloalkyl (such as a cyclopropyl group, a cyclobutyl group or a cyclopentyl group, a cyclohexyl group, etc. or the like), an aromatic (such as phenyl group, a naphthyl group, an anthracene group, a fluorenyl group, or the like), a heterocyclic (such as an epoxide group, a furyl group, a pyrrolyl group, a thiazolyl group, an oxazolyl group, an imidazolyl group, a pyridyl group, a pyrazinyl group, a pyrimidinyl group, a piperidinyl group, a pyrazolyl group, etc.), a hydroxyl, an alkoxy (such as a methoxy, an ethoxy or similar), an amine such as —NH₂, an alkylamine (such as methyl amine, ethyl amine propyl amine or the like), a dialkylamine (such as dimethyl amine, diisopropyl amine, etc.) an arylamine (such as phenyl amine, naphthyl amine etc.), a diarylamine (such as diphenyl amine or the like), an amide such as —CONH₂, an N-substituted amide (where substituent is an alkyl or an aryl group or carboxylic acid or an alkoxy carbonyl group, such as a methoxy carbonyl group, an ethoxy carbonyl group, a phenoxy carbonyl etc.), an alkyl sulfonyl (such as a methyl sulfonyl group, an ethyl sulfonyl group, a p-tolyl sulfonyl group, a cyclohexyl sulfonyl group, etc.), a ketone (such as methyl ketone, an ethyl ketone or a phenyl ketone, etc.), a sulfonamide, a nitrile, a nitro, a halogen (for example, F, Cl, Br or I), a trifluoromethyl, a trichloromethyl, a tribromomethyl, an acyl, or the like.

For example, in one embodiment, R₁ may comprise a coumarin, R₂ may be —H, and R₃ may comprise a fluorene. As a specific example, the molecule may have the following structure:

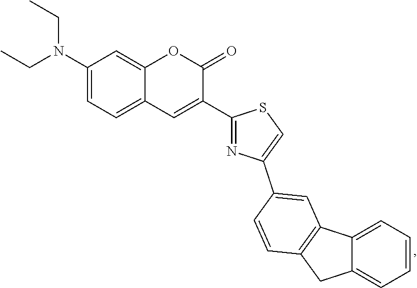

where R₅ is diethylamine, and R₄, R₆, R₇, R₈, R₉, R₁₀, R₁₁, R₁₂, R₁₃, R₁₄, R₁₅, and R₁₆ are hydrogen atoms.

In another embodiment, the 1,3-thiazole may be a 4-methyl-1,3-thiazole, e.g., having a structure:

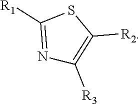

R₁ and R₂ may be as discussed above, while R₃ may be a methyl. For instance, R₁ may comprise a furan and/or R₂ may comprise an oxadiazole. As an example, in some embodiments, R₁ may be 2,3-dihydrobenzofuran, R₂ may be 2-(naphthalen-1-yl)-1,3,4-oxadiazole, and R₃ may be methyl.

In certain embodiments, R₁ may have a structure such as:

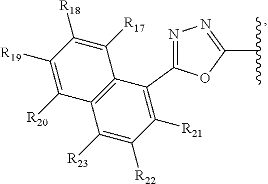

where R₁₇, R₁₈, R₁₉, R₂₀, R₂₁, R₂₂, and R₂₃ may each independently be —H or selected from an alkyl (such as a methyl group, an ethyl group, a propyl group, a butyl group or the like), a cycloalkyl (such as a cyclopropyl group, a cyclobutyl group or a cyclopentyl group, a cyclohexyl group, etc. or the like), an aromatic (such as phenyl group, a naphthyl group, an anthracene group, a fluorenyl group, or the like), a heterocyclic (such as an epoxide group, a furyl group, a pyrrolyl group, a thiazolyl group, an oxazolyl group, an imidazolyl group, a pyridyl group, a pyrazinyl group, a pyrimidinyl group, a piperidinyl group, a pyrazolyl group, etc.), a hydroxyl, an alkoxy (such as a methoxy, an ethoxy or similar), an amine such as —NH₂, an alkylamine (such as methyl amine, ethyl amine propyl amine or the like), a dialkylamine (such as dimethyl amine, diisopropyl amine, etc.) an arylamine (such as phenyl amine, naphthyl amine etc.), a diarylamine (such as diphenyl amine or the like), an amide such as —CONH₂, an N-substituted amide (where substituent is an alkyl or an aryl group or carboxylic acid or an alkoxy carbonyl group, such as a methoxy carbonyl group, an ethoxy carbonyl group, a phenoxy carbonyl etc.), an alkyl sulfonyl (such as a methyl sulfonyl group, an ethyl sulfonyl group, a p-tolyl sulfonyl group, a cyclohexyl sulfonyl group, etc.), a ketone (such as methyl ketone, an ethyl ketone or a phenyl ketone, etc.), a sulfonamide, a nitrile, a nitro, a halogen (for example, F, Cl, Br or I), a trifluoromethyl, a trichloromethyl, a tribromomethyl, an acyl, or the like.

In certain embodiments, R₁ may have a structure such as:

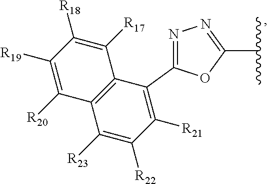

where R₁₇, R₁₈, R₁₉, R₂₀, R₂₁, R₂₂, and R₂₃ may each independently be —H or selected from an alkyl (such as a methyl group, an ethyl group, a propyl group, a butyl group or the like), a cycloalkyl (such as a cyclopropyl group, a cyclobutyl group or a cyclopentyl group, a cyclohexyl group, etc. or the like), an aromatic (such as phenyl group, a naphthyl group, an anthracene group, a fluorenyl group, or the like), a heterocyclic (such as an epoxide group, a furyl group, a pyrrolyl group, a thiazolyl group, an oxazolyl group, an imidazolyl group, a pyridyl group, a pyrazinyl group, a pyrimidinyl group, a piperidinyl group, a pyrazolyl group, etc.), a hydroxyl, an alkoxy (such as a methoxy, an ethoxy or similar), an amine such as —NH₂, an alkylamine (such as methyl amine, ethyl amine propyl amine or the like), a dialkylamine (such as dimethyl amine, diisopropyl amine, etc.) an arylamine (such as phenyl amine, naphthyl amine etc.), a diarylamine (such as diphenyl amine or the like), an amide such as —CONH₂, an N-substituted amide (where substituent is an alkyl or an aryl group or carboxylic acid or an alkoxy carbonyl group, such as a methoxy carbonyl group, an ethoxy carbonyl group, a phenoxy carbonyl etc.), an alkyl sulfonyl (such as a methyl sulfonyl group, an ethyl sulfonyl group, a p-tolyl sulfonyl group, a cyclohexyl sulfonyl group, etc.), a ketone (such as methyl ketone, an ethyl ketone or a phenyl ketone, etc.), a sulfonamide, a nitrile, a nitro, a halogen (for example, F, Cl, Br or I), a trifluoromethyl, a trichloromethyl, a tribromomethyl, an acyl, or the like.

In certain embodiments, R₂ may have a structure such as:

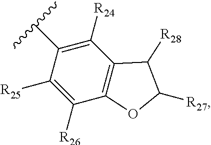

where R₂₄, R₂₅, R₂₆, R₂₇, and R₂₈ may each independently be —H or selected from an alkyl (such as a methyl group, an ethyl group, a propyl group, a butyl group or the like), a cycloalkyl (such as a cyclopropyl group, a cyclobutyl group or a cyclopentyl group, a cyclohexyl group, etc. or the like), an aromatic (such as phenyl group, a naphthyl group, an anthracene group, a fluorenyl group, or the like), a heterocyclic (such as an epoxide group, a furyl group, a pyrrolyl group, a thiazolyl group, an oxazolyl group, an imidazolyl group, a pyridyl group, a pyrazinyl group, a pyrimidinyl group, a piperidinyl group, a pyrazolyl group, etc.), a hydroxyl, an alkoxy (such as a methoxy, an ethoxy or similar), an amine such as —NH₂, an alkylamine (such as methyl amine, ethyl amine propyl amine or the like), a dialkylamine (such as dimethyl amine, diisopropyl amine, etc.) an arylamine (such as phenyl amine, naphthyl amine etc.), a diarylamine (such as diphenyl amine or the like), an amide such as —CONH₂, an N-substituted amide (where substituent is an alkyl or an aryl group or carboxylic acid or an alkoxy carbonyl group, such as a methoxy carbonyl group, an ethoxy carbonyl group, a phenoxy carbonyl etc.), an alkyl sulfonyl (such as a methyl sulfonyl group, an ethyl sulfonyl group, a p-tolyl sulfonyl group, a cyclohexyl sulfonyl group, etc.), a ketone (such as methyl ketone, an ethyl ketone or a phenyl ketone, etc.), a sulfonamide, a nitrile, a nitro, a halogen (for example, F, Cl, Br or I), a trifluoromethyl, a trichloromethyl, a tribromomethyl, an acyl, or the like.

For example, in one embodiment, R₁ may comprise a furan, R₂ may comprise an oxadiazole, and R₃ may be a methyl. As a specific example, the molecule may have the following structure:

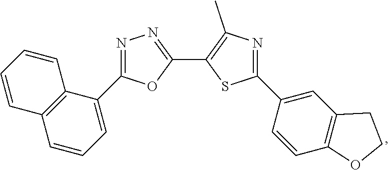 [SMILES: Cc1nc(-c2ccc3c(c2)CCO3)sc1-c1nnc(-c2cccc3ccccc23)o1]

where each of R₁₇, R₁₈, R₁₉, R₂₀, R₂₁, R₂₂, R₂₃, R₂₄, R₂₅, R₂₆, R₂₇, and R₂₈ are hydrogen atoms.

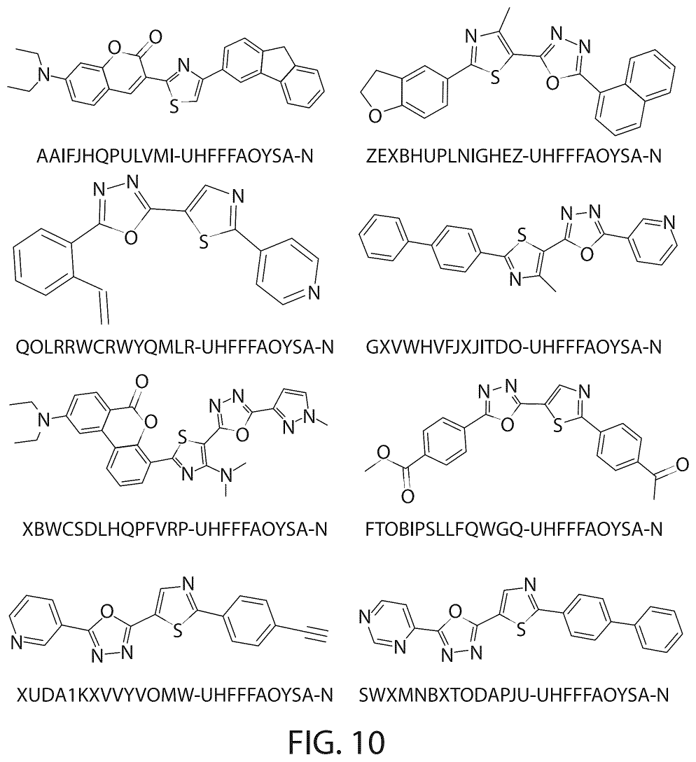

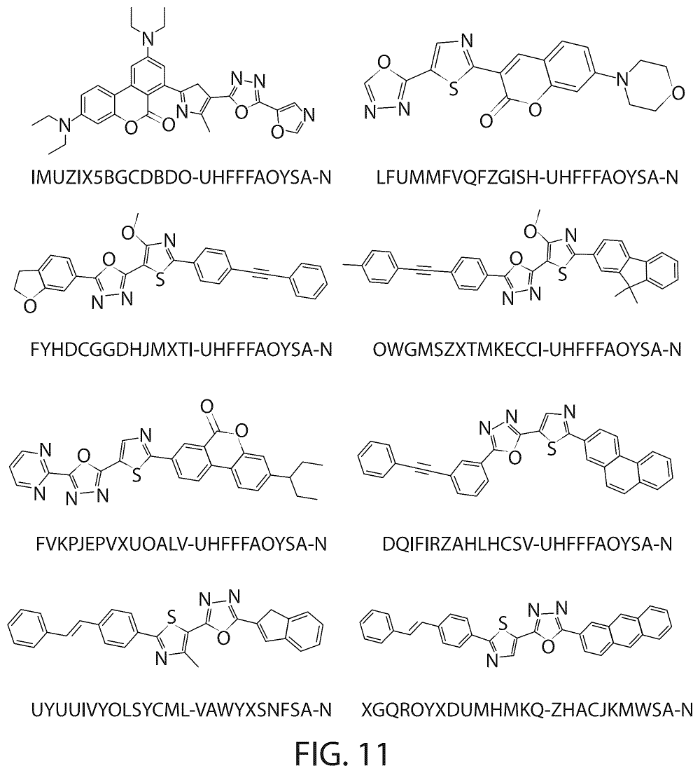

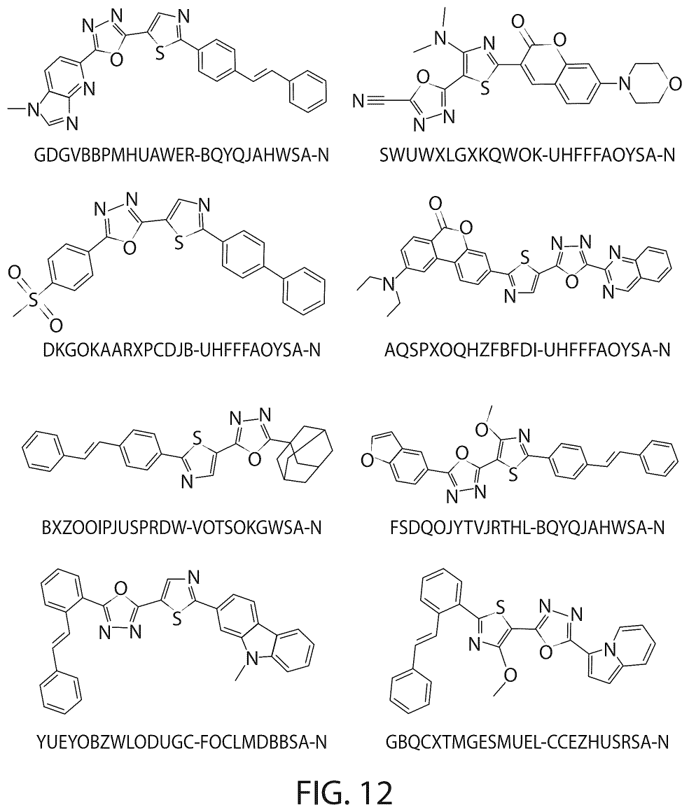

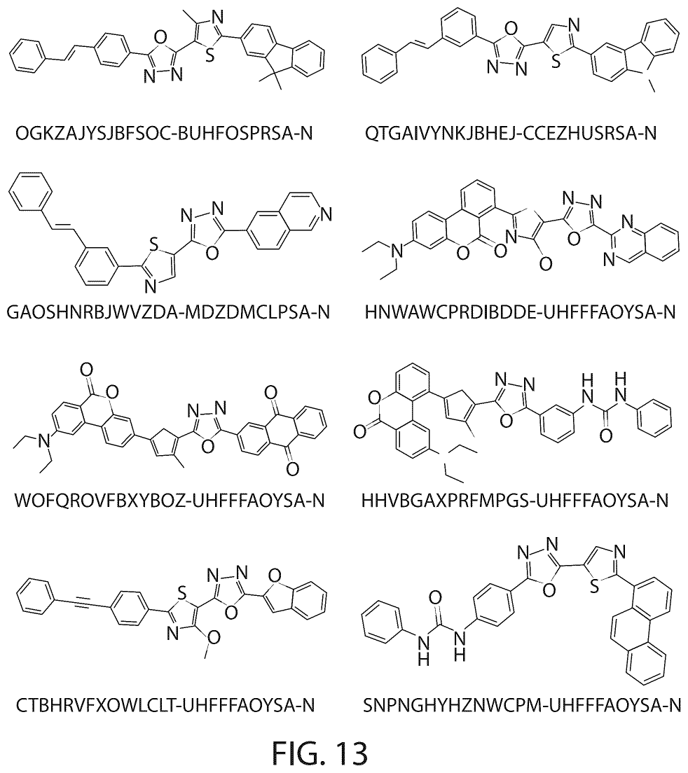

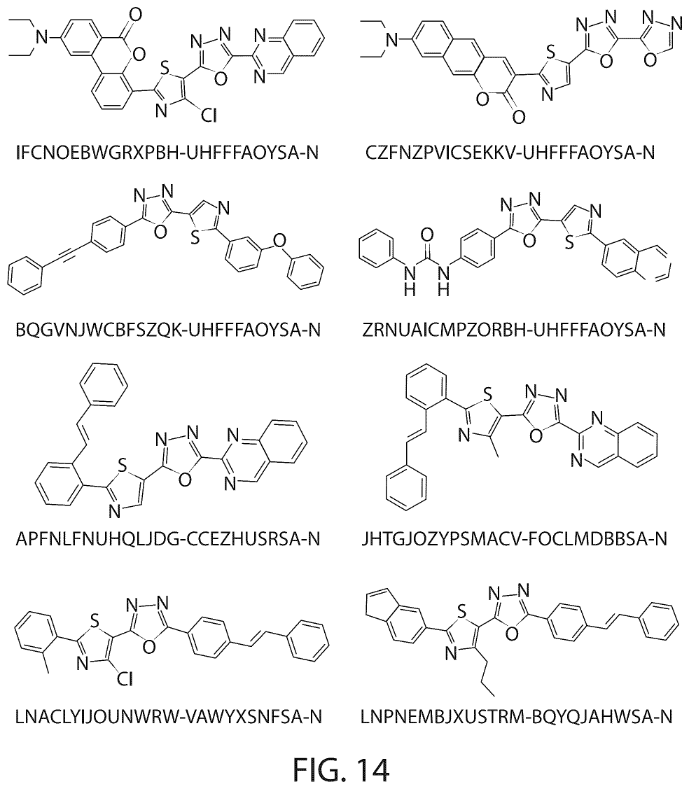

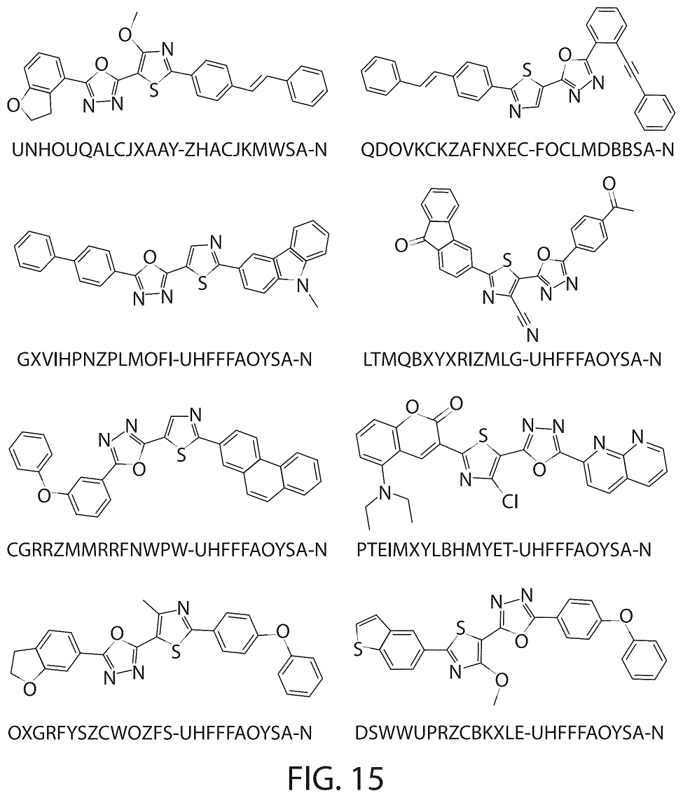

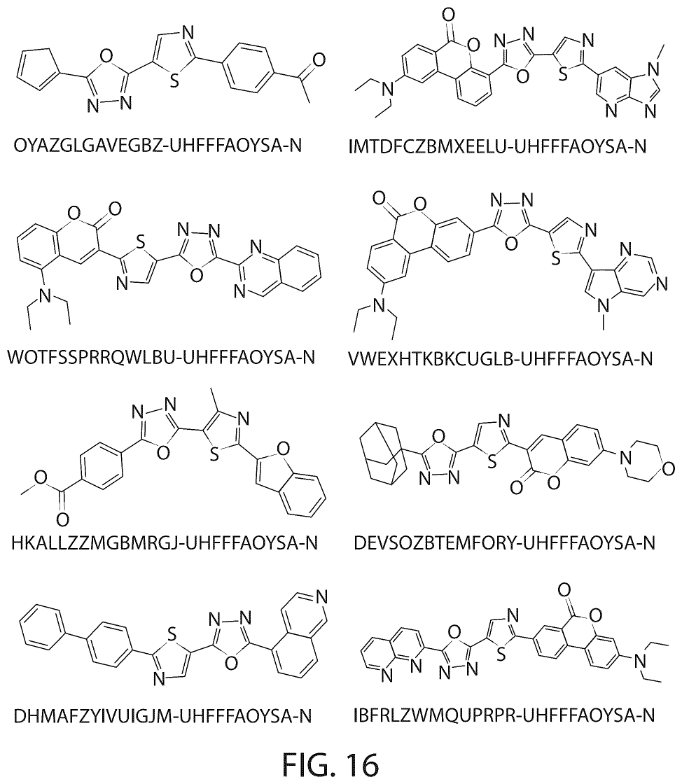

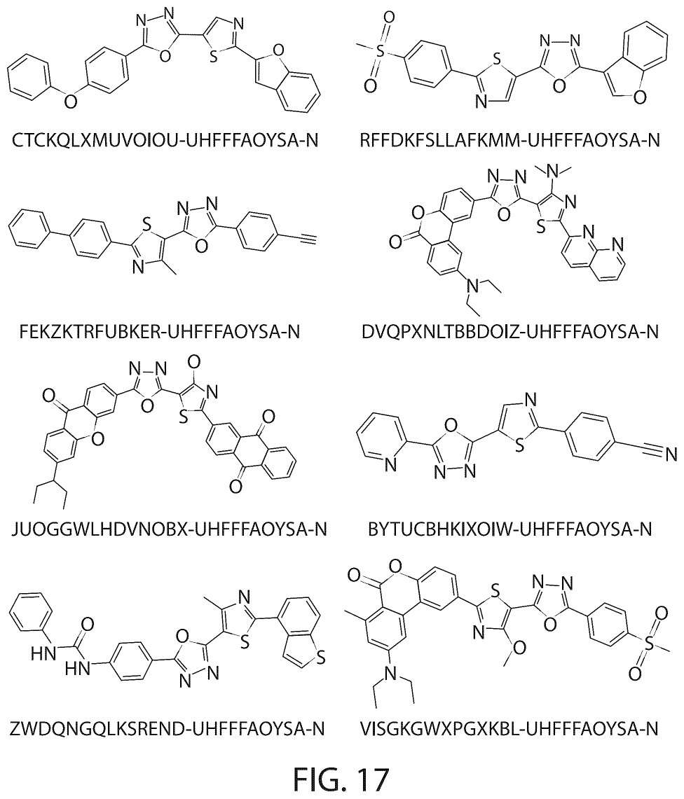

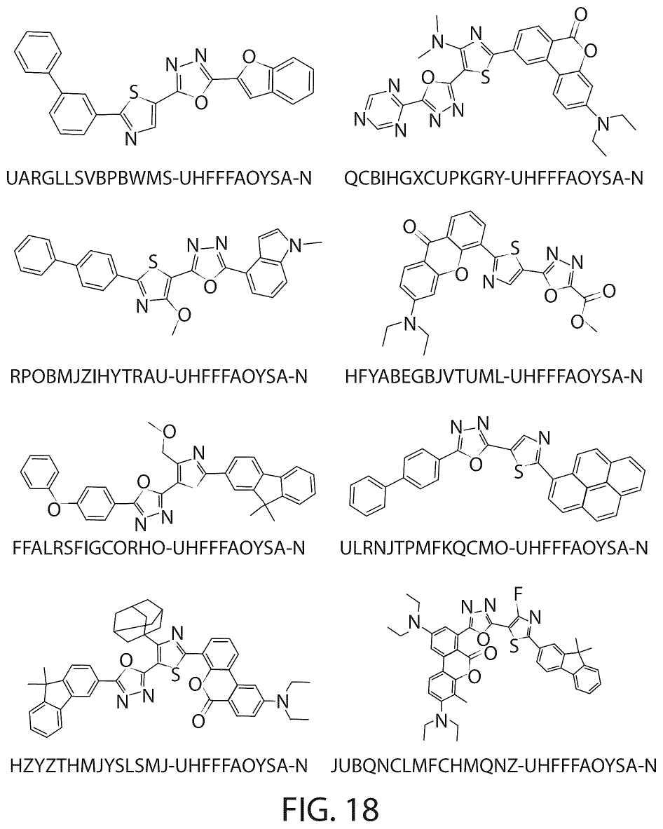

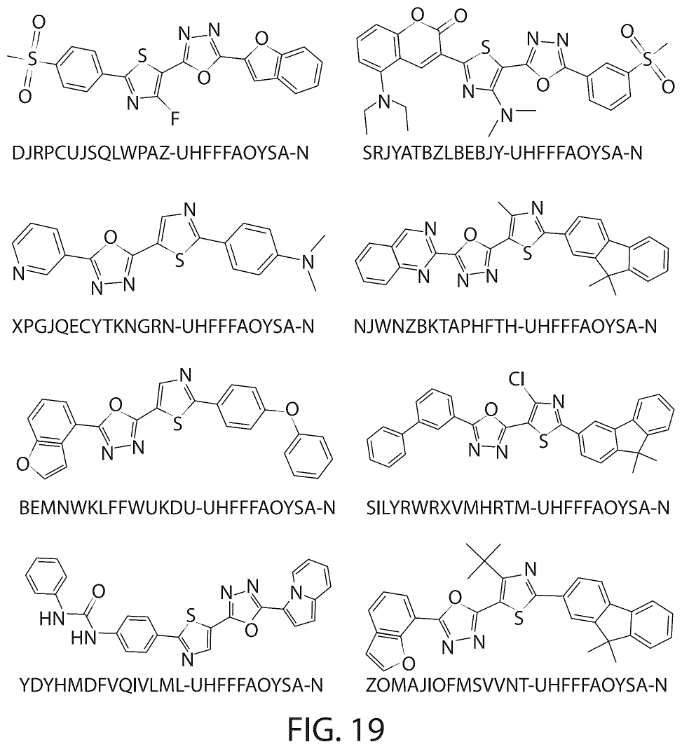

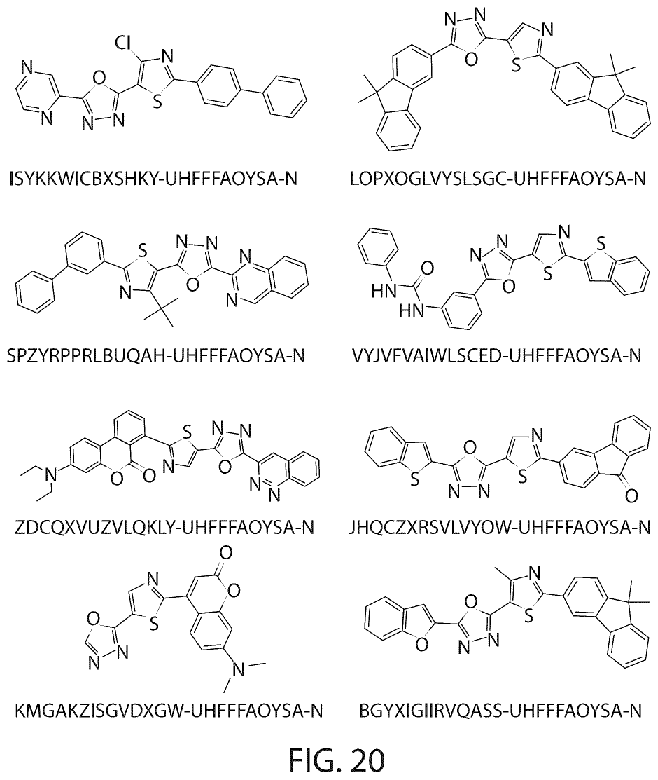

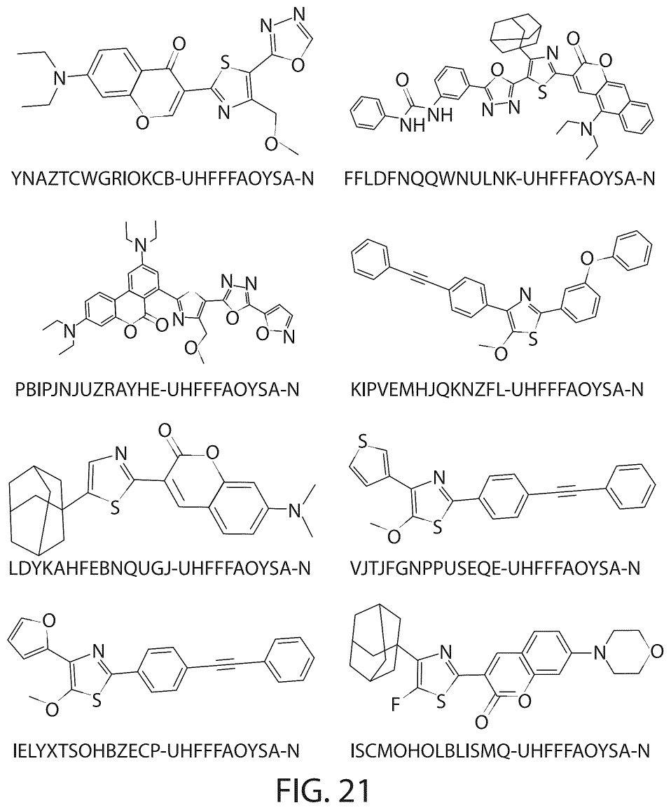

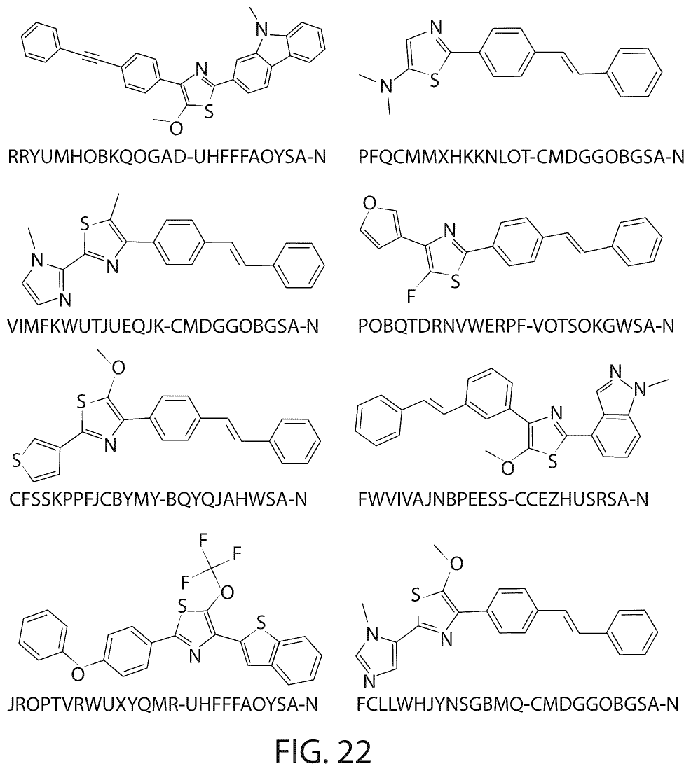

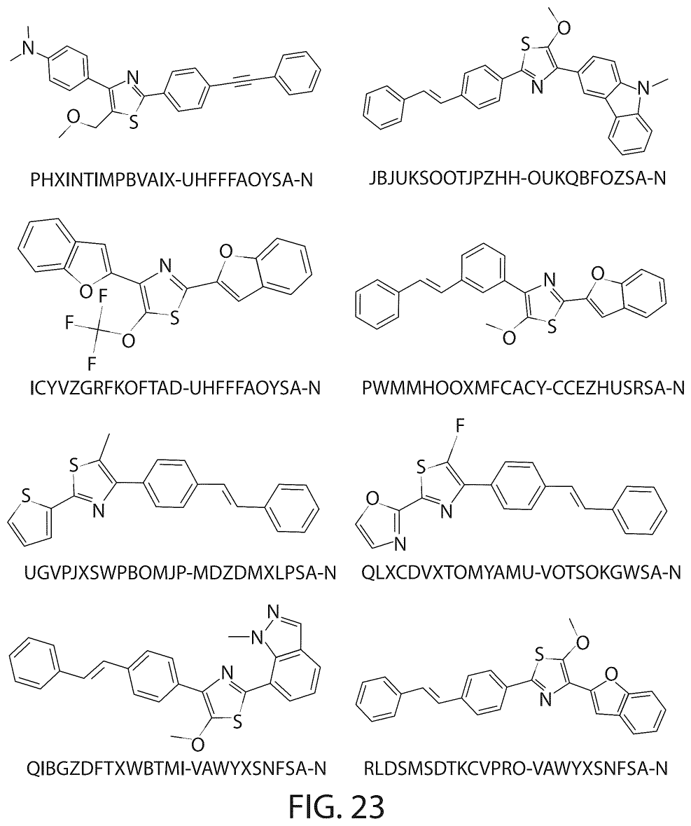

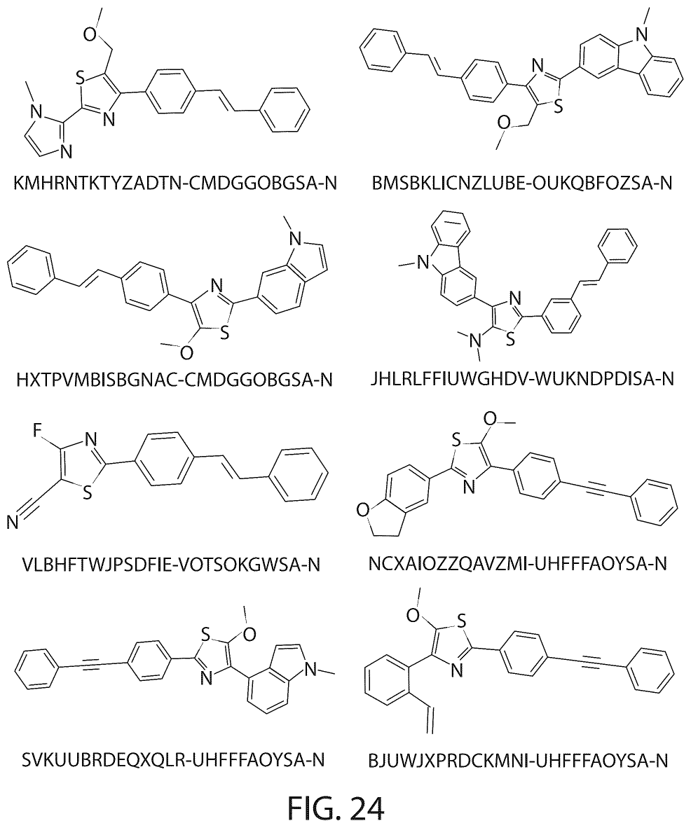

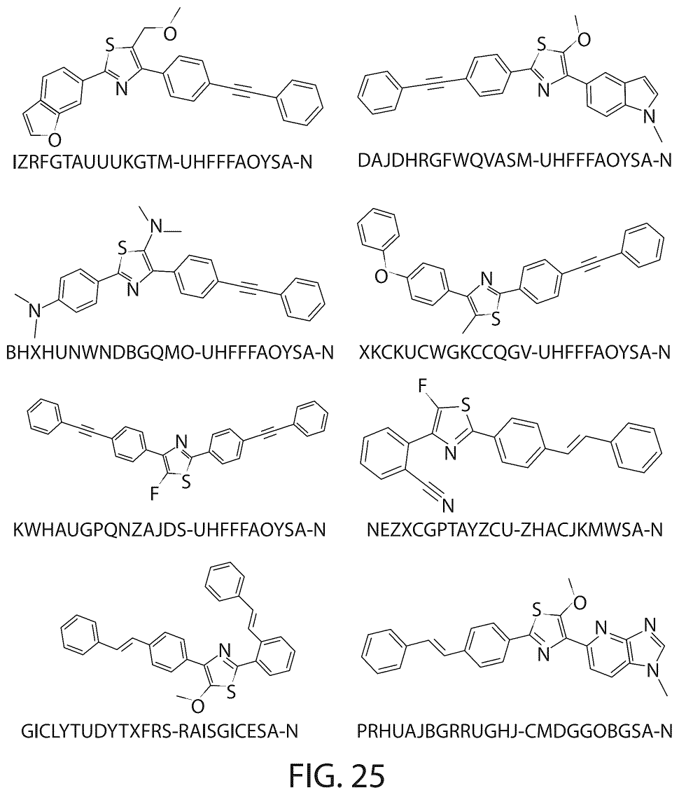

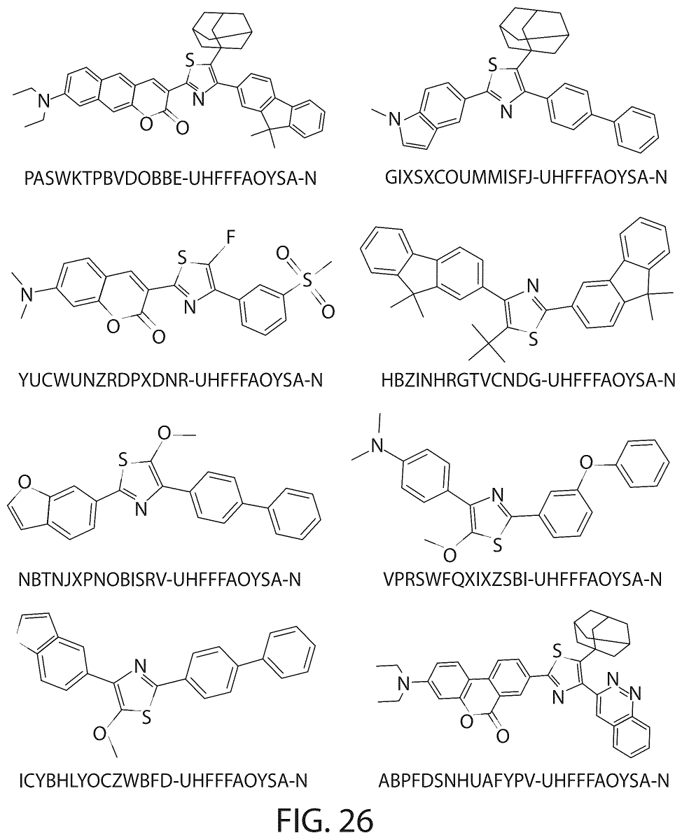

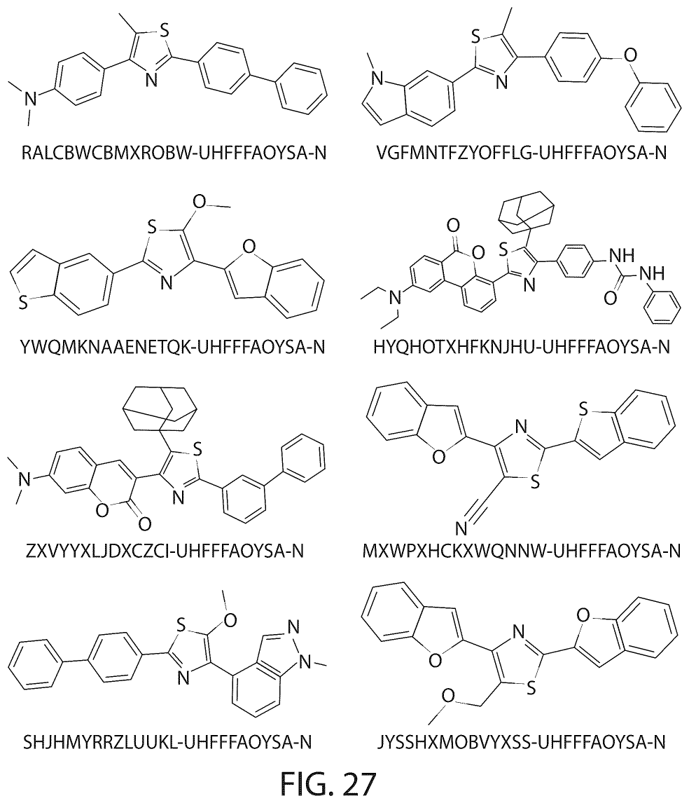

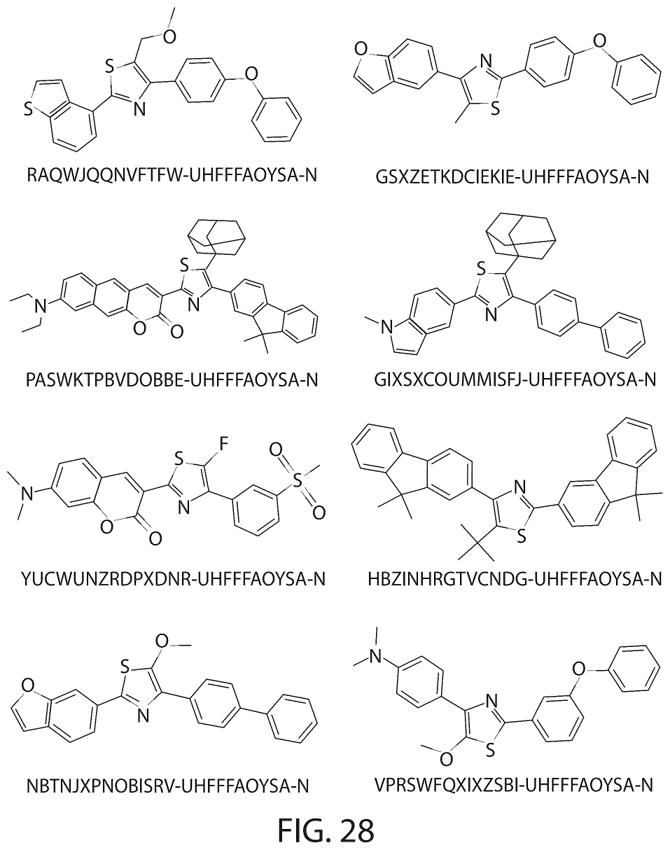

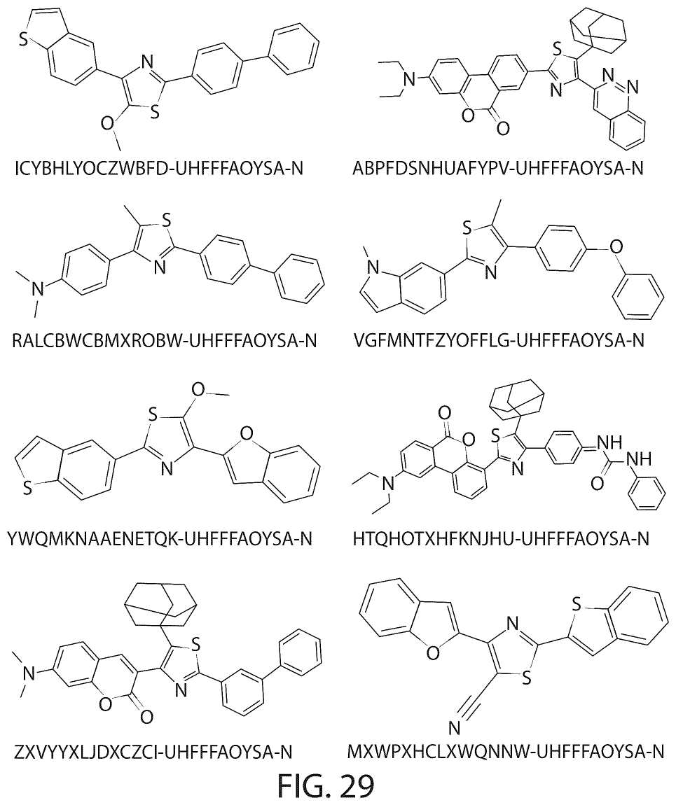

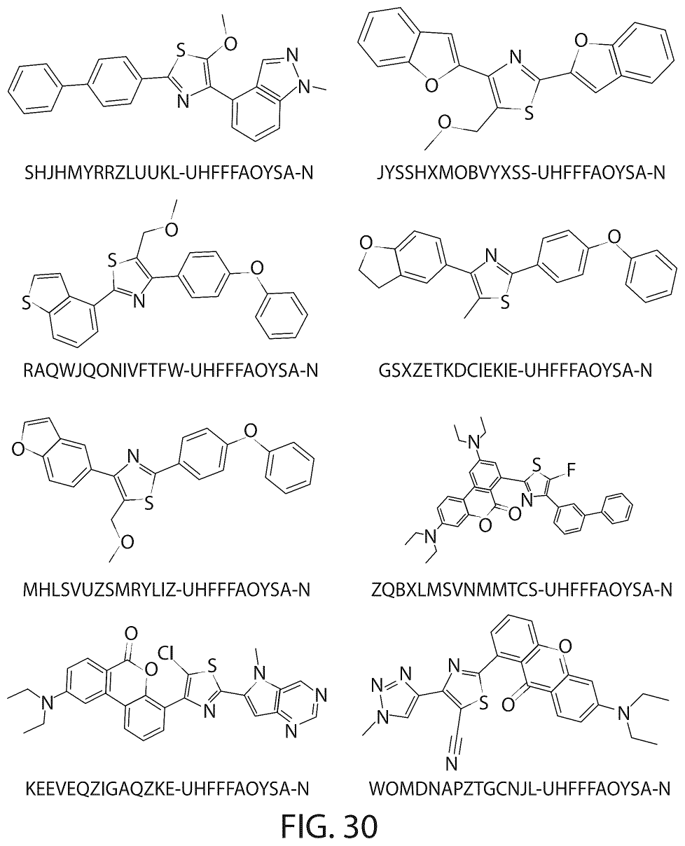

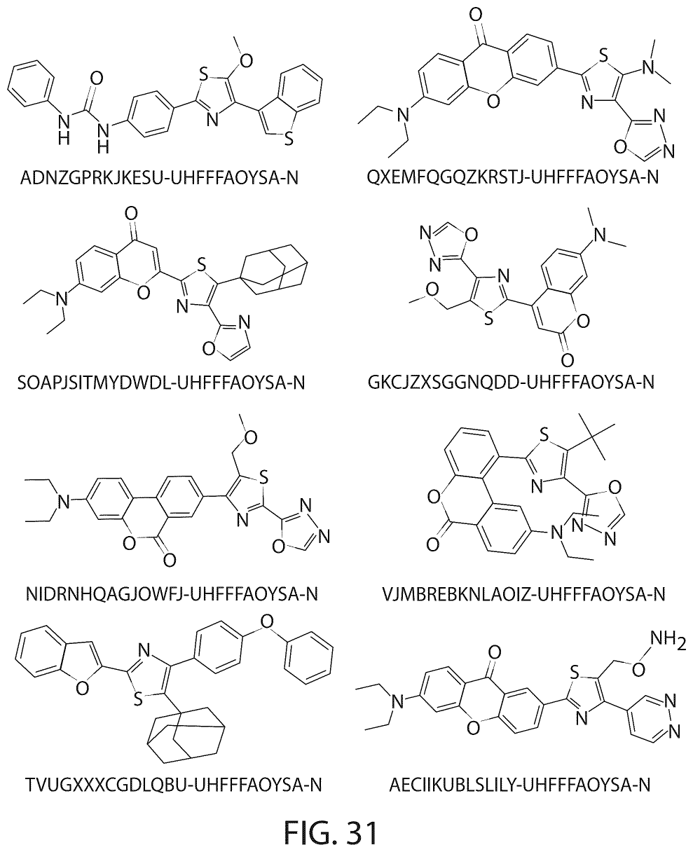

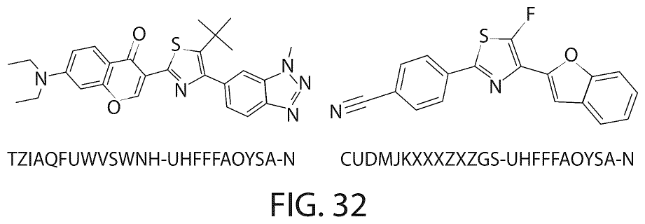

Still other non-limiting examples of potentially suitable molecules, including thiazoles such as 1,3-thiazoles, 4-methyl-1,3-thiazoles, etc., are shown in FIGS. 10-23. Each structure is identified by the first hash block (14 characters) of the InChIKey (International Chemical Identifier Key) of the molecule's structure (i.e., its connectivity information). Those of ordinary skill in the art will be familiar with the InChI system and its related InChIKey hashes.

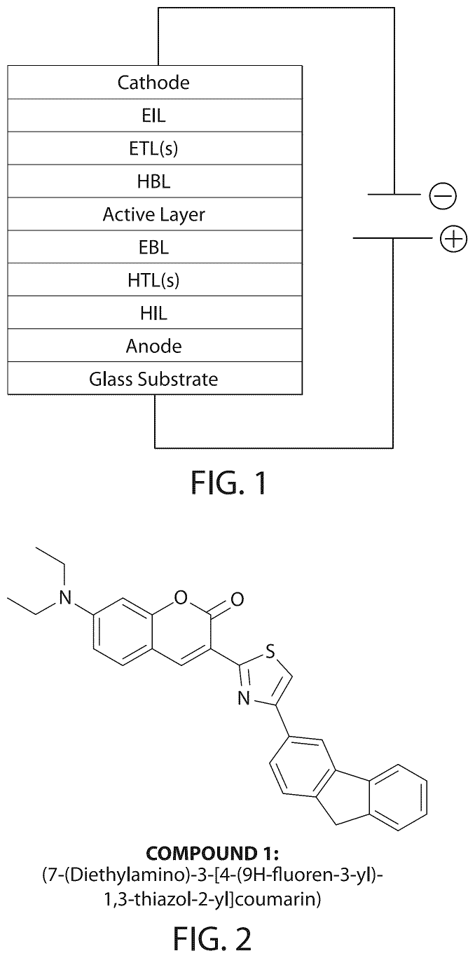

In some aspects, molecules such as any of those described herein may be contained within an electroluminescent device. Examples of such devices include, but are not limited to, indoor or outdoor luminaires, display devices, as well as molecular systems for digital information processors, optical recording, thermal writing displays, laser printers, infrared photography, or the like. An example of an electroluminescent device is shown in FIG. 1. However, it should be understood that this is by way of example only. In other embodiments, the electroluminescent device may have different structures or electrical configurations, etc. The electroluminescent device also need not be transparent or designed to allow light to pass through; an example of such a device is a transparent display.

In one set of embodiments, an electroluminescent device may comprise a cathode and anode, over which a voltage can be applied. Any suitable voltage can be applied, e.g., at least 1 V, at least 3 V, at least 5 V, at least 10 V, at least 30 V, at least 50 V, at least 100 V, etc. The voltage may be used to cause the flow of electrons from the cathode to the active layer and positive charges (holes) to flow from the anode to the active layer. As a non-limiting example, FIG. 1 shows an electroluminescent device comprising of both an anode and cathode with various organic layers between.

One or both of these electrodes may be substantially transparent in certain cases, e.g., to allow a substantial amount of light to pass through the electrode, at least in the visible range. For instance, an electrode may allow at least 50%, at least 70%, or at least 90% of the incident visible light to pass through. For instance, an electrode may be made from a substantially transparent material, such as indium tin oxide (ITO), ZnO:F, ZnO:Al, ZnO:Ga, ZnO:B, ZnO:In, In₂O₃:Sn, Cd₂SnO₄, SnO₂:Sb, conjugated polymers (such as PEDOT:PSS), silver nanowires, graphene, or other materials. However, in some embodiments, one or both of the electrodes may be made out of materials that are not substantially transparent, for example, metals such as platinum, gold, silver, or copper, or conductive non-metals such as carbon. In some cases, materials that are not substantially transparent may nonetheless be used, e.g., in embodiments where the electrode is substantially transparent, or where light is allowed to pass through the device; for example, the electrode may be formed as a mesh or other structure containing openings that allows at least some light to pass through. In addition, it should be understood that an electrode need not be substantially transparent in all embodiments, and that the electrodes within a device may have the same or different amounts of light transparency.

In some cases, the electroluminescent material is present in a separate region that is in direct contact with both electrodes. However, other configurations are possible. For example, there may be one or more other regions (e.g., layers) intervening between the layer containing the electroluminescent material and the cathode or anode. As an example, in FIG. 1, multiple layers of organic or inorganic materials may be deposited as thin layers between the active layer and an electrode. These include the EIL (electron injection layer), the ETL (electron transport layer), the HBL (hole blocking layer), the EBL (electron blocking layer), the HTL (hole transport layer), and the HIL (hole injection layer. However, it should be understood that not of these layers are required to be present, and in other embodiments, a variety of other arrangements of layers between the cathode and the anode are also possible. In other cases, the electroluminescent material may be in direct contact with the working electrode.

In some cases, the electroluminescent region may be relatively thin, for example, as a layer or a coating adjacent to an electrode. For instance, in some cases, the region may have a cross-sectional thickness of less than 1 micrometer, less than 500 nanometers, less than 100 nanometers, less than 50 nanometers, less than 25 nanometers, or less than 10 nanometers. Without wishing to be bound by any theory, it is believed that thinner regions may allow for more penetration of charges created at the electrodes. However, the electroluminescent region may also have sufficient thickness so as to exhibit light emission, e.g., when a voltage is applied.

For instance, in FIG. 1, the layer EBL is shown to be adjacent to electroluminescent region (active layer) containing the electroluminescent material, and when a voltage is applied, positive ions are able to flow from the anode to the electroluminescent region while simultaneously blocking the flow of electrons from reaching the anode. Similarly, the layer EBL is shown to be adjacent to an electroluminescent region (active layer) containing the electroluminescent material, and when a voltage is applied, negative ions are able to flow from the cathode to the electroluminescent region while simultaneously blocking the flow of holes from reaching the cathode. However, as noted above, in other cases, the electroluminescent region may not comprise additional layers, and the electroluminescent materials may be located adjacent to the electrodes within the device. Examples of hole transporting/injecting/blocking materials include, but are not limited to, tris(4-carbazoyl-9-ylphenyl)amine (TCTA), 1,1-bis[(di-4-tolylamino)phenyl]cyclohexane (TAPC), MoO₃ 1,4,5,8,9,11-hexaazatriphenylenehexacarbonitrile (HATCN), and the like. Examples of electron transporting/injecting/blocking materials include, but are not limited to, LiF, 1,3,5-Tris(3-pyridyl-3-phenyl)benzene (TmPyPB), tris(8-hydroxyquinoline)aluminum(III) (Alq3), bathophenanthroline (BPhen), and the like.

In addition, in certain embodiments, the electroluminescent device may also be contained within a suitable protective media. For example, the use of an encapsulant can have a beneficial impact on the lifespan of the device, reducing molecular oxygen and water from reaching electrochemically active sites during device operation. The protective media may be, for example, glass, plastics, UV-curable epoxies, or the like, and in certain embodiments, the protective media may be non-conductive. Examples of other protective media include, but are not limited to, polycarbonate, acrylic, polyvinyl chloride (PVC), polyethylene terephthalate glycol-modified (PETG), cyclic olefin copolymer, liquid silicon rubber, polyethylene, ionomer resin, transparent polypropylene, fluorinated ethylene propylene, styrene methyl methacrylate, styrene acrylonitrile resin, etc.

U.S. Provisional Patent Application Ser. No. 63/292,276, filed Dec. 21, 2021, entitled “Emitting Materials for Electroluminescent Devices and Other Applications,” is incorporated herein by reference in its entirety.

The following examples are intended to illustrate certain embodiments of the present disclosure, but do not exemplify the full scope of the disclosure.

### Example 1

One embodiment contemplates a molecule having formula C₂₉H₂₄N₂O₂S and exhibiting electroluminescence with emission in the green part of the visible spectrum in the range of 480 nm to 600 nm (FIG. 2). This structure is identified herein as COMPOUND 1.

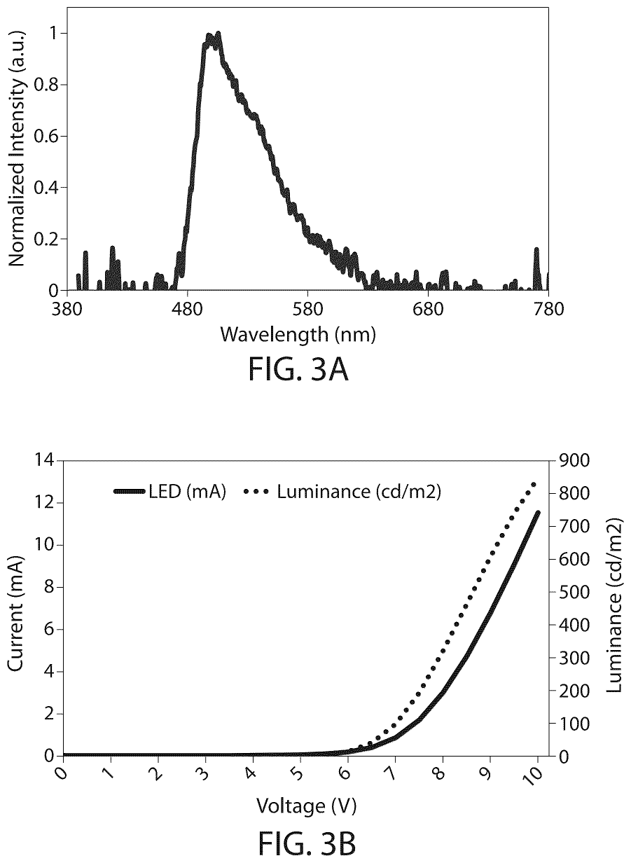

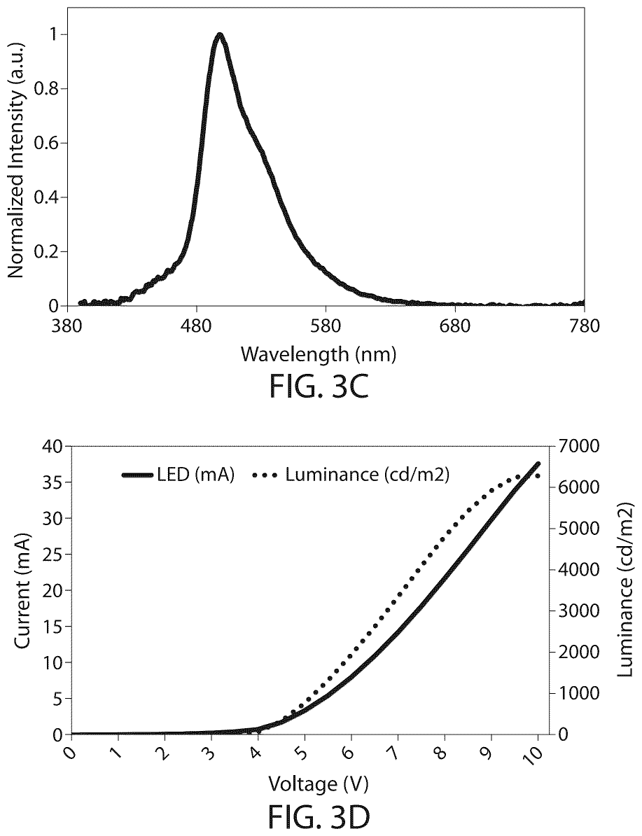

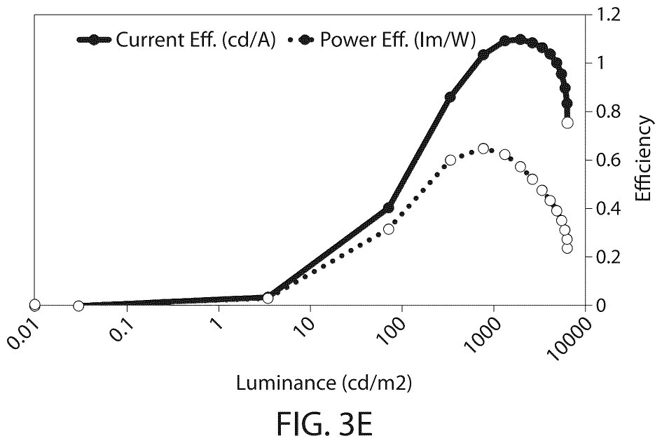

FIGS. 3A and 3B respectively show the electroluminescence spectrum and JVL (current density/voltage/luminance) curves for COMPOUND 1, while FIGS. 3C and 3D respectively show the electroluminescence spectrum and JVL (current density/voltage/luminance) curves in a solution-processed device. FIG. 3E show efficiency curves for COMPOUND 1.

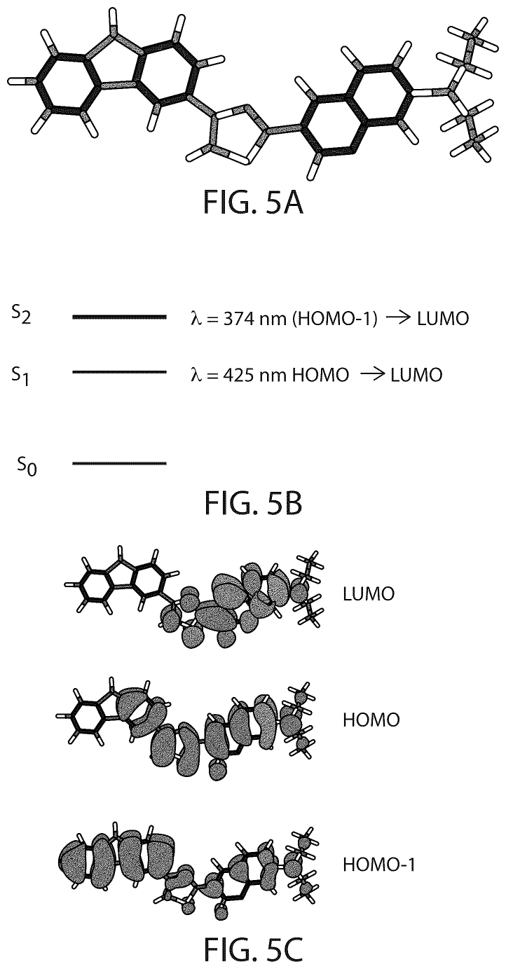

FIG. 5A illustrates the molecular structure of a thiazole-based green emitter, COMPOUND 1. FIG. 5B shows schematic diagram of energy levels with corresponding wavelengths and contributions of molecular orbitals computed using a quantum chemistry software. FIG. 5C shows the computed frontier electronic orbitals for the molecule shown in FIG. 5A.

### Example 2

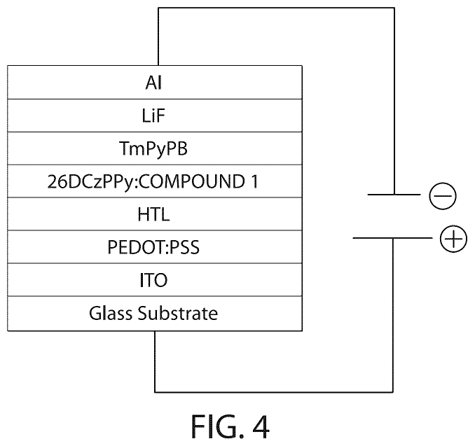

One non-limiting example of a device is shown in FIG. 4, showing a schematic architecture of an embodiment of the device using COMPOUND 1. This device comprises:
1. A glass substrate
2. A transparent electrode, for example, indium tin oxide (ITO)
3. A hole transport material, for example, a semiconducting polymer such as PEDOT:PSS, or an organic molecule, for example, 1,4,5,8,9,11-hexaazatriphenylenehexacarbonitrile (HATCN)
4. An electroluminescent OLED dispersed in a host matrix, for example, 2,6-bis(3-(carbazol-9-yl)phenyl)pyridine (26DCzPPy):COMPOUND 1 (5 wt %)
5. An electron transport material, for example, 1,3,5-tris(3-pyridyl-3-phenyl)benzene (TmPyPB)
6. An electron injection material, for example, lithium fluoride
7. A top electrode, for example, aluminum.

### Example 3

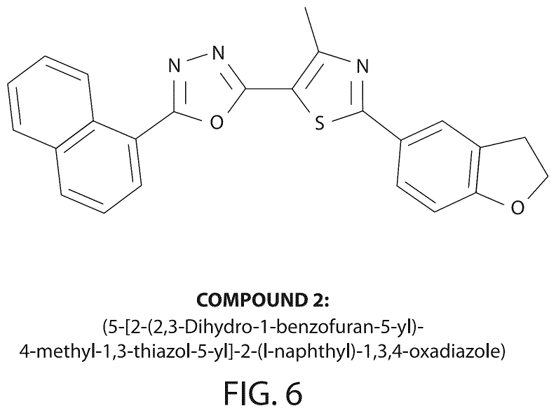

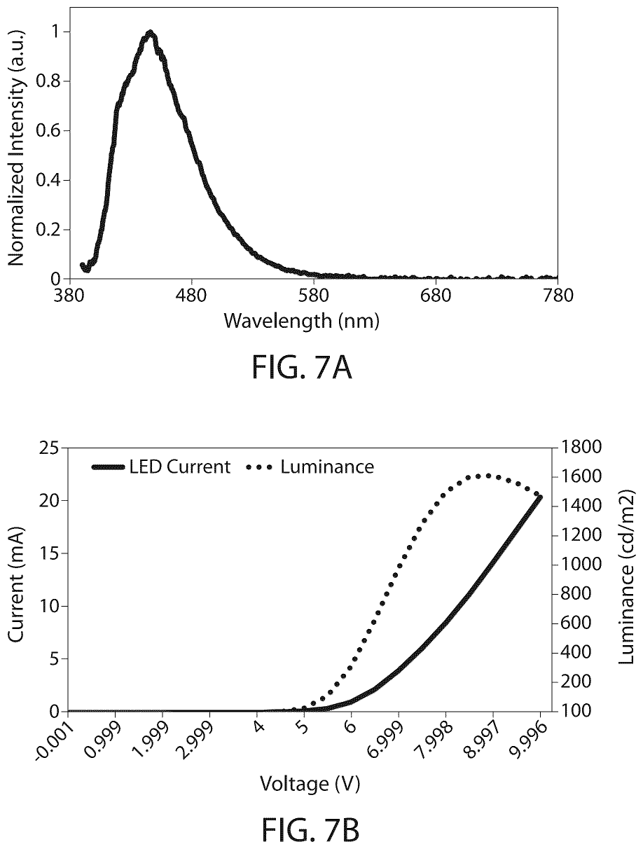

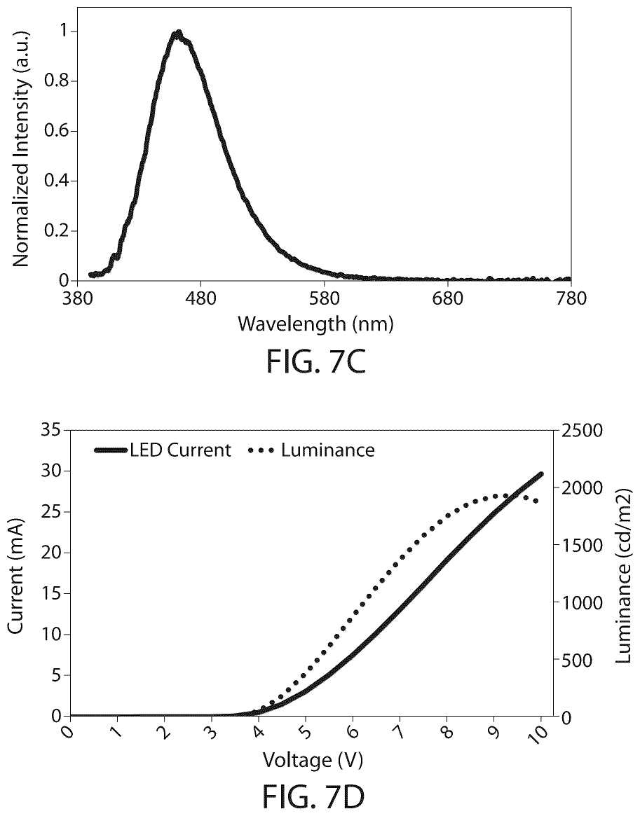

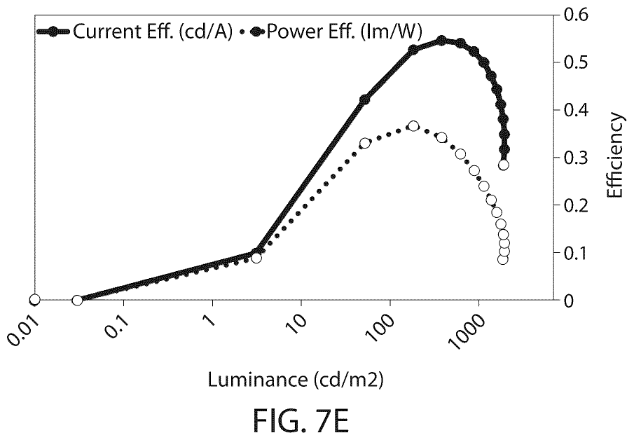

As another example, a compound identified herein as COMPOUND 2 is studied in this example. FIG. 6 shows the structure of COMPOUND 2. FIGS. 7A and 7B respectively show the electroluminescence spectrum and JVL curves for COMPOUND 2. FIGS. 7C and 7D respectively show the electroluminescence spectrum and JVL (current density/voltage/luminance) curves in a solution-processed device. FIG. 7E show efficiency curves for COMPOUND 2.

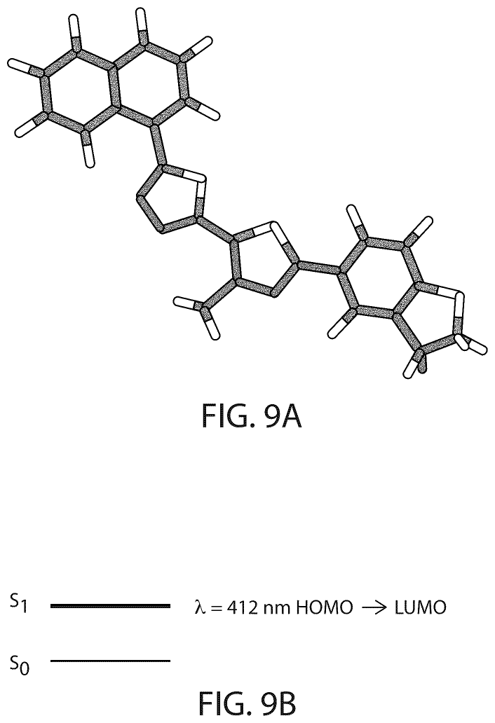

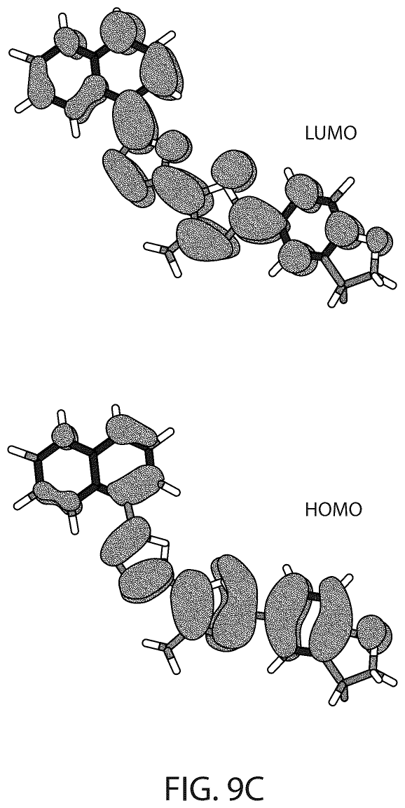

FIG. 9A shows the molecular structure of a thiazole-based blue emitter, COMPOUND 2. FIG. 9B shows a schematic diagram of energy levels with corresponding wavelengths and contributions of molecular orbitals computed using a quantum chemistry software. FIG. 9C shows the computed frontier electronic orbitals for the molecule shown in FIG. 9A.

### Example 4

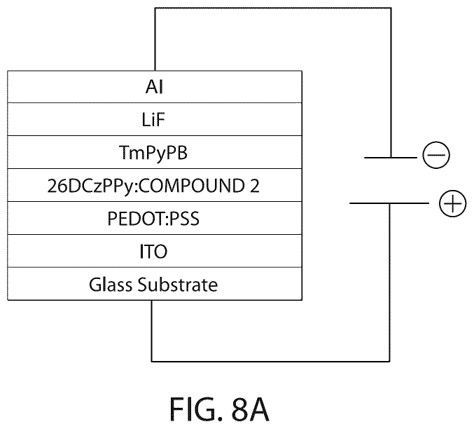

One non-limiting example of a device is shown in FIG. 8, showing a schematic architecture of an embodiment of the device using COMPOUND 2. This device comprises:
1. A glass substrate
2. A transparent electrode, for example, indium tin oxide (ITO)
3. A hole transport material, for example, a semiconducting polymer such as PEDOT:PSS, or organic molecule, for example, 1,4,5,8,9,11-hexaazatriphenylenehexacarbonitrile (HATCN)
4. An electroluminescent OLED dispersed in a host matrix, for example, 2,6-bis(3-(carbazol-9-yl)phenyl)pyridine (26DCzPPy):COMPOUND 2 (5 wt %)
5. An electron transport material, for example, 1,3,5-tris(3-pyridyl-3-phenyl)benzene (TmPyPB)
6. An electron injection material, for example, lithium fluoride
7. A top electrode, for example, aluminum.

### Example 5

This example illustrates one technique for making COMPOUND 1, in accordance with one embodiment. In this example, applying the Hantzsch condensation, the COMPOUND 1 can be synthesized from the acylbromide and coumarin thioamide under basic conditions in dimethylformamide as described in Takechi, et al., Chem. Pharm. Bull, 48:1702-1710, 2000.

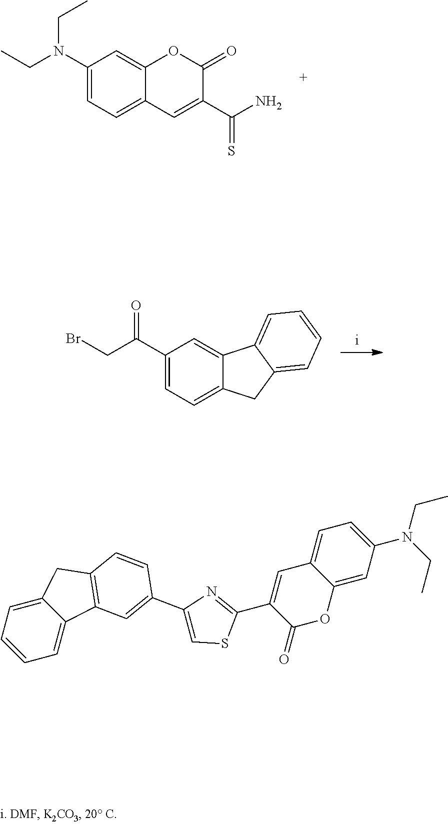 [SMILES: CCN(CC)c1ccc2cc(C(N)=S)c(=O)oc2c1 ; O=C(CBr)c1ccc2c(c1)-c1ccccc1C2 ; CCN(CC)c1ccc2cc(-c3nc(-c4ccc5c(c4)-c4ccccc4C5)cs3)c(=O)oc2c1]

The acylbromide is available in a single step from its precursor ketone and the thioamide, which can be made from the corresponding 7-diethylaminocoumarine-3-carboxamide with Lawesson's reagent, makes this synthetic route generally applicable to many OLED molecules discussed herein, e.g., having a thiazole.

### Example 6

This example illustrates a method of preparing thiazoles from a 2,4-disubstituted thiazoles via palladium catalyzed arylation, generally based on Roger, et al., J. Org. Chem., 74(3):1179-1186, 2009:

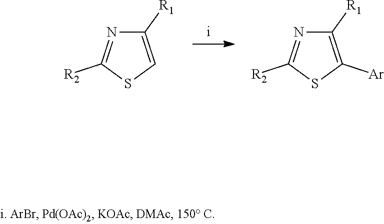

Other 2,4,5-trisubstituted thiazoles, may also be synthesized via the widely used Hantzsch condensation reaction.

From a common intermediate which can be obtained in three linear steps to the thiazole ring system followed by selective functionalization of the bis-halo thiazole provides derivatives of the core structure COMPOUND 1:

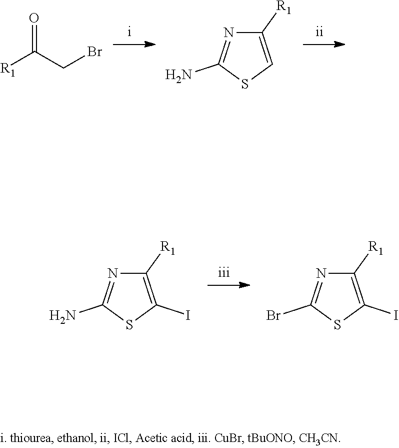

### Example 7

This example illustrates one certain techniques for making COMPOUND 2, in accordance with one embodiment. The synthesis of the linked heterocyclic systems can be constructed in many ways, starting with one heterocycle, and building the second one from functional groups on the first heterocycle. In COMPOUND 2, for example, the oxadiazole ring can be synthesized starting from commercially available thiazole-2-carboxylic acid derivative. Converting the carboxylic acid to the acid chloride may be followed by a reaction with hydrazine to produce the acylhydrazone. Acid chloride derived from 1-naphthoic acid can react with the acylhydrazone to form the diacylhydrazone which cyclizes in presence of POCl₃, as shown here:

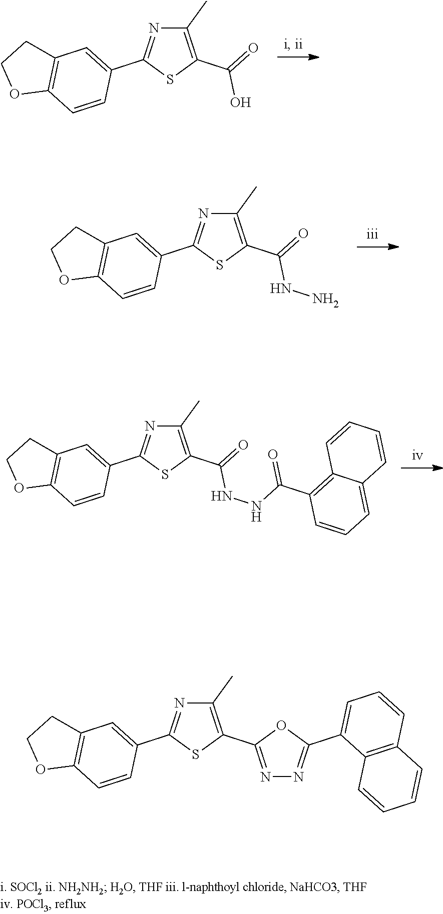 [SMILES: Cc1nc(-c2ccc3c(c2)CCO3)sc1C(=O)O ; Cc1nc(-c2ccc3c(c2)CCO3)sc1C(=O)NN ; Cc1nc(-c2ccc3c(c2)CCO3)sc1C(=O)NNC(=O)c1cccc2ccccc12 ; Cc1nc(-c2ccc3c(c2)CCO3)sc1-c1nnc(-c2cccc3ccccc23)o1]

### Example 8

This example illustrates an approach towards the synthesis of molecules structurally similar to COMPOUND 2 core, following the route as shown below. Direct arylation of the oxadiazole core (prepared from corresponding arylhydrazide and triethylorthoformate; see generally Hedouin, et al., Org. Lett., 20(19):6027-6032, 2018) under copper catalyzed conditions can be used to synthesize molecules having a linked heterocyclic core, e.g., as described in Kawano, et al., Org. Lett., 11(14):3072-3075, 2009)

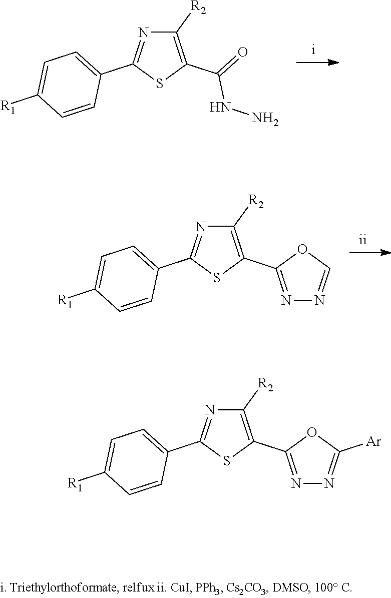

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
applying voltage to an electroluminescent material comprising a 1,3-thiazole to cause the electroluminescent material to exhibit light emission, wherein the 1,3-thiazole comprises a structure: 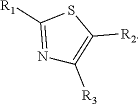
wherein each of R₁, R₂, and R₃ is independently an aromatic group or —H such that only one of R₂ and R₃ is —H.

2. The method of claim 1, wherein the 1,3-thiazole comprises a structure: 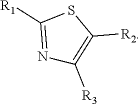
wherein R₁ is a coumarin, R₃ is a fluorene, and R₂ is —H.

3. The method of claim 2, wherein the coumarin has a structure: 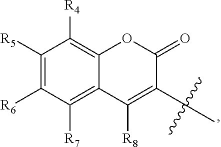
wherein R₄, R₅, R₆, R₇, and R₈ are independently selected from the group consisting of —H, an alkyl, a cycloalkyl, an aromatic, a heterocyclic, an imidazolyl, a pyridyl, a pyrazinyl, a pyrimidinyl, a piperidinyl, a pyrazolyl, a hydroxyl, an alkoxy, an amine, an alkylamine, a dialkylamine, an arylamine, an amide, an N-substituted amide, an alkyl sulfonyl, a ketone, a sulfonamide, a nitrile, a nitro, a halogen, a trifluoromethyl, a trichloromethyl, a tribromomethyl, or an acyl.

4. The method of claim 3, wherein R₅ is diethylamine, and R₄, R₆, R₇, and R₈ are each —H.

5. The method of claim 2, wherein the fluorene has a structure: 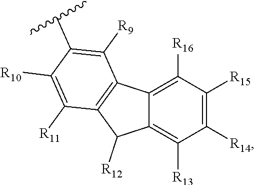
wherein R₉, R₁₀, R₁₁, R₁₂, R₁₃, R₁₄, R₁₅, and R₁₆ are independently selected from the group consisting of —H, an alkyl, a cycloalkyl, an aromatic, a heterocyclic, an imidazolyl, a pyridyl, a pyrazinyl, a pyrimidinyl, a piperidinyl, a pyrazolyl, a hydroxyl, an alkoxy, an amine, an alkylamine, a dialkylamine, an arylamine, an amide, an N-substituted amide, an alkyl sulfonyl, a ketone, a sulfonamide, a nitrile, a nitro, a halogen, a trifluoromethyl, a trichloromethyl, a tribromomethyl, or an acyl.

6. The method of claim 5, wherein R₉, R₁₀, R₁₁, R₁₂, R₁₃, R₁₄, R₁₅, and R₁₆ are each —H.

7. The method of claim 2, wherein the 1,3-thiazole comprises a structure:

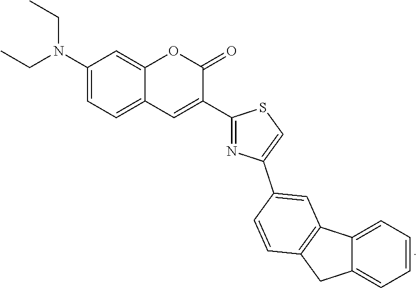 [SMILES: CCN(CC)c1ccc2cc(-c3nc(-c4ccc5c(c4)-c4ccccc4C5)cs3)c(=O)oc2c1]

8. (canceled)

9. The method of claim 1, wherein the 1,3-thiazole comprises a structure: 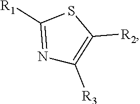
wherein R₁ is a furan, R₂ is an oxadiazole, and R₃ is methyl.

10. The method of claim 9, wherein the oxadiazole has a structure: 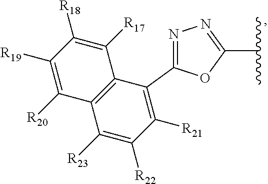
wherein R₁₇, R₁₈, R₁₉, R₂₀, R₂₁, R₂₂, and R₂₃ are independently selected from the group consisting of —H, an alkyl, a cycloalkyl, an aromatic, a heterocyclic, an imidazolyl, a pyridyl, a pyrazinyl, a pyrimidinyl, a piperidinyl, a pyrazolyl, a hydroxyl, an alkoxy, an amine, an alkylamine, a dialkylamine, an arylamine, an amide, an N-substituted amide, an alkyl sulfonyl, a ketone, a sulfonamide, a nitrile, a nitro, a halogen, a trifluoromethyl, a trichloromethyl, a tribromomethyl, or an acyl.

11. The method of claim 10, wherein R₁₇, R₁₈, R₁₉, R₂₀, R₂₁, R₂₂, and R₂₃ are each —H.

12. The method of claim 9, wherein the oxadiazole is 2-(naphthalen-1-yl)-1,3,4-oxadiazole.

13. The method of claim 9, wherein the furan has a structure: 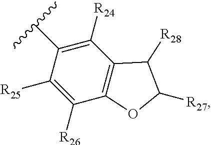
wherein R₂₄, R₂₅, R₂₆, R₂₇, and R₂₈ are independently selected from the group consisting of —H, an alkyl, a cycloalkyl, an aromatic, a heterocyclic, an imidazolyl, a pyridyl, a pyrazinyl, a pyrimidinyl, a piperidinyl, a pyrazolyl, a hydroxyl, an alkoxy, an amine, an alkylamine, a dialkylamine, an arylamine, an amide, an N-substituted amide, an alkyl sulfonyl, a ketone, a sulfonamide, a nitrile, a nitro, a halogen, a trifluoromethyl, a trichloromethyl, a tribromomethyl, or an acyl.

14. The method of claim 13, wherein R₂₄, R₂₅, R₂₆, R₂₇, and R₂₈ are each —H.

15. (canceled)

16. The method of any one of claim 9, wherein the 1,3-thiazole comprises a structure:

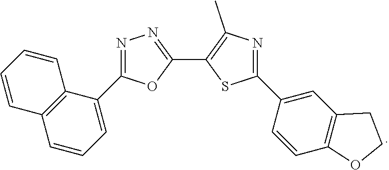 [SMILES: Cc1nc(-c2ccc3c(c2)CCO3)sc1-c1nnc(-c2cccc3ccccc23)o1]

17. The method of claim 1, wherein the 1,3-thiazole is selected from FIGS. 10-23.

18. The method of claim 1, wherein the 1,3-thiazole is selected from the group consisting of the following molecules, each identified by a first hash block of an InChIKey representation of the molecule: AAIFIJHQPULVMI, ZEXBHUPLNIGHEZ, QOLRRWCRWYQMLR, GXVWHVFJXJITDO, XBWCSDLHQPFVRP, FTOBIPSLLFQWGQ, XUDAQKXVVYVOMW, SWXMNBXTODAPJU, IMUZIXSBGCDBDO, LFUMMFVQFZGISH, LFUMMFVQFZGISH, OWGMSZXTMKECCI, FVKPJEPVXUOALV, DQIFIRZAHLHCSV, UYUUIVYOLSYCML, XGQROYXDUMHMKQ, GDGVBBPMHUAWER, SWUVVXLGXKQWOK, DKGOKAARXPCDJB, AQSPXOQHZFBFDI, BXZOOIPJUSPRDW, FSDQOJYTVJRTHL, YUEYOBZWLODUGC, GBQCXTMGESMUEL, OGKZAJYSJBFSOG, QTGAIVYNKJBHEJ, GAOSHNRBJWVZDA, HNWAWCPRDIBDDE, WOFQROVFBXYBOZ, HHVBGAXPRFMPGS, CTBHRVFXOWLCLT, SNPNGHYHZNWCPM, IFCNOEBWGRXPBH, CZFNZPVICSEKKV, BQGVNJWCBFSZQK, ZRNUAICMPZORBH, APFNLFNUHQLJDG, JHTGJOZYPSMACV, LNACLYIJOUNWRW, LNPNEMBJXUSTRM, UNHOUQALCJXAAY, QDOVKCKZAFNXEC, GXVIHPNZPLMOFI, LTMQBXYXRIZMLG, CGRRZMMRRFNWPW, PTEIMXYLBHMYET, OXGRFYSZCWOZFS, DSWWUPRZCBKXLE, OYAZGLJGAVEGBZ, IMTDFCZBMXEELU, WOTFSSPRRQWLBU, VWEXHTKBKCUGLB, HKALLZZMGBMRGJ, DEVSOZBTEMFORY, DHMAFZYIVUIGJM, IBFRLZWMQUPRPR, CTCKQLXMUVOIOU, RFFDKFSLLAFKMM, FEKZKTRIFUBKER, DVQPXNLTBBDOIZ, JUOGGWLHDVNOBX, IBYTUCBHKIXOIW, ZWDQNGQLKSREND, VISGKGWXPGXKBL, UARGLLSVBPBWMS, QCBIHGXCUPKGRY, RPOBMJZIHYTRAU, HFYABEGBJVTUML, FFALRSFIGCORHO, ULRNJTPMFKQCMO, HZYZTHMJYSLSMJ, JUBQNCLMFCHMQN, DJRPCUJSQLWPAZ, SRJYATBZLBEBJY, XPGJQECYTKNGRN, NJWNZBKTAPHFTH, BEMNWKLFFWUKDU, SILYRWRXVMHRTM, YDYHMDFVQIVLML, ZOMAJIOFMSVVNT, ISYKKWICBXSHKY, LOPXOGLVYSLSGC, SPZYRPPRLBUQAH, VYJVFVAIWLSCED, ZDCQXVUZVLQKLY, JHQCZXRSVLVYOW, KMGAKZISGVDXGW, BGYXIGIIRVQASS, YNAZTCWGRIOKCB, FFLDFNQQENULNK, PBIPJNJUZRAYHE, KIPVEMHJQKNZFL, LDYKAHFEBNQUGJ, VJTJFGNPPUSEQE, IELYXTSOHBZECP, ISCMOHOLBLISMQ, RRYUMHOBKQOGAD, PFQCMMXHKKNLOT, VIMFKWUTJUEQJK, POBQTDRNVWERPF, CFSSKPPFJCBYMY, FWVIVAJNBPEESS, JROPTVRWUXYQMR, FCLLWHJYNSGBMQ, PHXINTIMPBVAIX, JBJUKSOOTJPZHH, ICYVZGRFKOFTAD, PWMMHOOXMFCACY, UGVPJXSWPBOMJP, QLXCDVXTOMYAMU, QIBGZDFTXWBTMI, RLDSMSDTKCVPRO, KMHRNTKTYZADTN, BMSBKLICNZLUBE, HXTPVMBISBGNAC, JHLRLFFIUWGHDV, VLBHFTWJPSDFIE, NCXAIOZZQAVZMI, SVKUUBRDEQXQLR, BJUWJXPRDCKMNI, IZRFGTAUUUKGTM, DAJDHRGFWQVASM, BHXHUNWNDBGQMO, XKCKUCWGKCCQGV, KWHAUGPQNZAJDS, NEZXCGPTAYIZCU, GICLYTUDYTXFRS, PRHUAJBGRRUGHJ, PASWKTPBVDOBBE, GIXSXCOUMMISFJ, YUCWUNZRDPXDNR, HBZINHRGTVCNDG, NBTNJXPNOBISRV, VPRSWFQXIXZSBI, ICYBHLYOCZWBFD, ABPFDSNHUAFYPV, RALCBWCBMXROBW, VGFMNTFZYOFFLG, YWQMKNAAENETQK, HYQHOTXHFKNJHU, ZXVYYXLJDXCZCI, MXWPXHCLXWQNNW, SHJHMYRRZLUUKL, JYSSHXMOBVYXSS, RAQWJQONIVFTFW, GSXZETKDCIEKIE, PASWKTPBVDOBBE, GIXSXCOUMMISFJ, YUCWUNZRDPXDNR, HBZINHRGTVCNDG, NBTNJXPNOBISRV, VPRSWFQXIXZSBI, ICYBHLYOCZWBFD, ABPFDSNHUAFYPV, RALCBWCBMXROBW, VGFMNTFZYOFFLG, YWQMKNAAENETQK, HYQHOTXHFKNJHU, ZXVYYXLJDXCZCI, MXWPXHCLXWQNNW, SHJHMYRRZLUUKL, JYSSHXMOBVYXSS, RAQWJQONIVFTFW, GSXZETKDCIEKIE, MHLSVUZSMRYLIZ, ZQBXLMSVNMMTCS, KEEVEQZIGAQZKE, WOMDNAPZTGCNJL, ADNZGPRKIJKESU, QXEMFQGQZKRSTJ, SOAPJSITMYDWDL, GKCJZXSGGINQDD, NIDRNHQAGJOWFJ, VJMBREBKNLAOIZ, TVUGXXXCGDLQBU, AECIIKUVLSLILY, TZIAQFUWVSWNHI, and CUDMJKXXXZXZGS.

19. The method of claim 1, wherein the electroluminescent material exhibits light emission.

20. (canceled)

21. The method of claim 1, wherein the electroluminescent material exhibits light emission at a wavelength of between 300 nm and 400 nm.

22. The method of claim 1, wherein the electroluminescent material exhibits light emission at a wavelength of between 450 nm and 600 nm.

23-24. (canceled)

25. The method of claim 1, wherein the electroluminescent material is contained within an electroluminescent device.

26. The method of claim 25, wherein the electroluminescent device is a display.

27-28. (canceled)

29. The method of claim 25, wherein the electroluminescent device is an automotive luminaire.

30-67. (canceled)
